(19) United States
(12) Patent Application Publication
(10) Pub. No.: US 2021/0295963 A1
(43) Pub. Date: Sep. 23, 2021

(54) REAL-TIME INTERACTIVE DIGITAL EMBODIMENT OF A PATIENT

(71) Applicant: BuddhiMed Technologies Pvt. Ltd., Bangalore (IN)

(72) Inventors: Ajay Bakshi, New Delhi (IN); Rohit Gupta, Bangalore (IN)

(21) Appl. No.: 16/822,148

(22) Filed: Mar. 18, 2020

(51) Int. Cl. G16H 10/60; G16H 40/63; G16H 30/20

(52) U.S. Cl. CPC G16H 10/60; G16H 30/20; G16H 40/63

(57) ABSTRACT

Aspects of the disclosure relate to real-time interactive digital embodiment of a patient. A computing platform may retrieve, via the communication interface and from a medical repository, an electronic health record associated with a patient. Then, the computing platform may extract, from the electronic health record, data indicative of: a plurality of patient features indicative of one or more of a physical feature or a mental state associated with the patient, and a plurality of health attributes of the patient. The computing platform may then configure a digital embodiment of the patient to: display the plurality of patient features, and display information associated with the plurality of health attributes. Subsequently, the computing platform may render, via a graphical user interface of a computing device, the digital embodiment of the patient.

### BACKGROUND

Aspects of the disclosure relate to deploying digital data processing systems to provide fast, reliable, knowledge-based, and real-time information about doctor-patient interactions. In particular, one or more aspects of the disclosure relate to a real-time representation of medical information associated with a patient via an interactive digital embodiment of the patient.

The doctor-patient interaction is central to the universe of healthcare operations. Other aspects, including, for example, investigations, may depend on laboratory and radiology services, that depend on the doctor-patient interaction. Similarly, the doctor-patient interaction is central to prescriptions that may need the pharmaceutical industry, and interventions like surgery that may need devices, instruments, and hospitals. Also, for example, a need to train doctors and other healthcare professionals may also emanate from this interaction. These doctor-patient interactions may happen in the out-patient (OP) setting (e.g., a consultation), a day-care setting (e.g., a dialysis or minor operative procedures like a biopsy) or an in-patient setting (e.g., the Operations Theatre or the Intensive Care Unit). The OP setting is the most high-volume transaction and it is universal in nature.

The OP interaction consists of four categories of sub-activities that include: 1) reviewing old information about the patient, 2) eliciting new information (through interview and examination), 3) decision making about the diagnosis, further investigations, and/or therapy, and 4) performing procedures and writing prescriptions to implement the decisions made at step 3. Generally, such activities may not be based on technology. For example, devices used in sub-activity 2) may include a stethoscope, which was invented in 1816, and the sphygmomanometer (for measuring blood pressure), that was invented in 1881. Although various versions of Electronic Health Records (EHRs) may be available, such EHR solutions may not provide physicians with intelligent summaries and alerts indicative of medical factors that may be of significant importance to a patients' care.

Several areas of medicine have witnessed a proliferation of technology. In particular, human understanding of diseases and their underlying pathologies has been enhanced, laboratory tests and radiology investigations to confirm such pathologies, and therapies available to remedy many of these diseases (e.g., surgical, or medical treatments) has advanced considerably. Such advancement of knowledge has also led to fragmentation in nomenclature and terms used to describe the various diseases, pathologies, investigations, and therapies. For example, such nomenclature and terms may be different in different regions of the world. In some examples, different hospitals within the same country may use different terms to describe the same thing. For example, a surgery to remove the gall bladder may be called any of the following: Cholecystectomy, Gallbladder excision, Removal of Gall bladder and Excision of Gall bladder among other things. Similarly, a lab test to evaluate the blood might be called a Hemogram, Complete Blood Count or CBC in different healthcare systems even within the same geography.

Medical coding systems have been developed to standardize such diverse nomenclatures and some of these systems like, for example, SNOMED, ICD-10, LOINC etc. have been adopted widely, and now form the basis of many financial transactions in the healthcare industry. Such coding systems may generally be based on hierarchical knowledge tree structures. For example, approximately 75,000 line-items in the LOINC system that codify laboratory and radiology investigations may be summarized into 24 top level chapters (e.g., Microbiology, Hematology, Serology etc.) with multiple levels of sub-categories being present between the top-level chapters and the lower level granular tests. However, insights based on such coding systems, and the standardized nomenclatures they represent, have not been brought to the physicians. Therefore, physicians may have to spend an inordinate amount of time to study paper-based medical records that patients bring to their clinics, analyze them, and gain insights from new information to arrive at a decision. However, there may be several challenges. For example, physicians may have no way of reviewing the information in a summarized manner so that they may focus on the relevant observations. Also, for example, even after spending a lot of time, physicians may not feel confident that they have not missed an important finding. In particular, such “missed observations” may be a significant contribution toward medical errors.

### SUMMARY

A system of one or more computers may be configured to perform particular operations or actions by virtue of having software, firmware, hardware, or a combination of them installed on the system that in operation causes or cause the system to perform the actions. One or more computer programs may be configured to perform particular operations or actions by virtue of including instructions that, when executed by data processing apparatus, cause the apparatus to perform the actions. One general aspect includes a computing platform having at least one processor, a communication interface, and memory may retrieve, via the communication interface and from a medical repository, an electronic health record associated with a patient. Then, the computing platform may extract, from the electronic health record, data indicative of: a plurality of patient features indicative of one or more of a physical feature or a mental state associated with the patient, and a plurality of health attributes of the patient. The computing platform may then configure a digital embodiment of the patient to: display the plurality of patient features, and display information associated with the plurality of health attributes. Subsequently, the computing platform may render, via a graphical user interface of a computing device, the digital embodiment of the patient. Other embodiments of this aspect include corresponding computer systems, apparatus, and computer programs recorded on one or more computer storage devices, each configured to perform the actions of the methods.

Implementations may include one or more of the following features. In some embodiments, the computing platform may configure the digital embodiment by detecting an interaction of the patient with a medical provider. Then, the computing platform may apply a timestamp to the digital embodiment of the patient, where the timestamp may be indicative of a time of the interaction.

In some embodiments, the computing platform may configure the digital embodiment by configuring, for each interaction of the patient with the medical provider, a temporal version of the digital embodiment, where the temporal version may be indicative of the electronic health record at the time of the interaction.

In some embodiments, the computing platform may render the digital embodiment by rendering, via the graphical user interface of the computing device, a plurality of temporal versions of the digital embodiment arranged in chronological order, where each temporal version of the plurality of temporal versions may be associated with the time of the interaction.

In some embodiments, the computing platform may detect, via user interaction with the graphical user interface, an indication of a particular time of the interaction. Then, the computing platform may provide, via the graphical user interface, the temporal version of the digital embodiment corresponding to the particular time.

In some embodiments, the digital embodiment may be a three-dimensional rendering of the patient. In some embodiment, the computing device may detect, via the graphical user interface, a user interaction indicative of a movement associated with the digital embodiment. Then, the computing device may cause the digital embodiment to perform the indicated movement.

In some embodiments, the computing device may be associated with the patient, and the computing platform may perform the rendering based on one or more of a sub-plurality of the plurality of patient features, and a sub-plurality of the plurality of health attributes. Then, the computing platform may provide the rendered digital embodiment to the computing device associated with the patient.

In some embodiments, the computing device may be associated with a medical professional with an access to the electronic health record of the patient, and the computing platform may perform the rendering based on a sub-plurality of the plurality of patient features. Then, the computing platform may provide the rendered digital embodiment to the computing device associated with the medical professional.

In some embodiments, the computing platform may configure the digital embodiment by identifying, for a particular health attribute of the plurality of health attributes, a particular location on or around the digital embodiment corresponding to the particular health attribute. Then, the computing platform may display the information associated with the particular health attribute at the particular location on or around the digital embodiment. In some embodiments, the information associated with the particular health attribute may be located in a hierarchical level of a hierarchical structure of medical information.

In some embodiments, the computing platform may detect, via user interaction with the digital embodiment, a user selection of a hierarchical level. Then, the computing platform may display, via the digital embodiment, the information associated with the particular health attribute, where the display information corresponds to the selected hierarchical level.

In some embodiments, the computing platform may configure the digital embodiment by detecting a change in the electronic health record of the patient. Then, the computing platform may update, based on the detected change, the rendering of the digital embodiment.

In some embodiments, the computing platform may configure the digital embodiment by extracting the plurality of patient features from a visual image or a video of the patient. Then, the computing platform may configure the digital embodiment based on the extracted features.

In some embodiments, the computing platform may configure the digital embodiment by animating a face of the digital embodiment to display one or more facial expressions. In some embodiments, the computing platform may animate the face by identifying, for each facial expression, a collection of facial muscles associated with the facial expression. Then, the computing platform may associate, for the collection of facial muscles, a set of rules that mimic the facial expression on the face of the digital embodiment.

In some embodiments, the computing platform may receive information related to a state of mind for the patient. Then, the computing platform may associate a facial expression with the state of mind. Subsequently, the computing platform may configure a face of the digital embodiment for the patient to display the associated facial expression.

In some embodiments, the computing platform may associate, with the patient, a wellness score indicative of the patient's well-being. Then, the computing platform may associate, for the digital embodiment, a body posture with the wellness score. Subsequently, the computing platform may configure the body posture of the digital embodiment for the patient to display the wellness score. In some embodiments, the computing platform may receive, via the graphical user interface and from the patient, the wellness score.

In some embodiments, the computing platform may associate, with each health attribute of the plurality of health attributes, an attribute score, and where the wellness score may be an aggregate of attribute scores.

In some embodiments, the computing platform may determine, for each health attribute of the plurality of health attributes, a temporal trend. Then, the computing platform may associate, for the digital embodiment, a body posture with the temporal trend. Subsequently, the computing platform may configure the body posture of the digital embodiment for the patient to display the temporal trend.

In some embodiments, the physical feature may include one or more of hair color, eye color, eye movement, voice, gait, items of clothing, clothing accessories, and facial expression.

In some embodiments, the computing platform may update, in real-time, the rendering of the digital embodiment.

In some embodiments, the computing platform may associate, with each organ of the patient and based on the electronic health record, a health score indicative of a health of the organ. Then, the computing platform may associate, with each health score, a color scheme. Subsequently, the computing platform may determine, for each organ of the patient, a region of the digital embodiment associated with the organ. Then, the computing platform may display, for the region and based on the health score associated with the organ, a color from the color scheme.

In some embodiments, the computing platform may determine, based on health scores associated with organs of the patient, an aggregate health score for the patient. Then, the computing platform may determine, for the aggregate health score, an aggregate color for the digital embodiment, where the aggregate color may be a combination of colors associated with the health scores.

In some embodiments, the computing platform may detect, from the electronic health record, presence of a medical implant in the patient. Then, the computing platform may determine, from the electronic health record, a physical location of the medical implant. Subsequently, the computing platform may configure the interactive digital embodiment of the patient to display an indication of the medical implant at a location, on the digital embodiment, that corresponds to the physical location.

Implementations of the described techniques may include hardware, a method or process, or computer software on a computer-accessible medium.

These features, along with many others, are discussed in greater detail below.

### BRIEF DESCRIPTION OF THE DRAWINGS

The present disclosure is illustrated by way of an example and not limited in the accompanying figures in which like reference numerals indicate similar elements and in which:

FIGS. 1A and 1B depict an illustrative computing environment for a real-time interactive digital embodiment of a patient in accordance with one or more example embodiments;

FIG. 2 depicts an illustrative flow diagram for a real-time interactive digital embodiment of a patient in accordance with one or more example embodiments;

FIG. 3 depicts another illustrative flow diagram for a real-time interactive digital embodiment of a patient in accordance with one or more example embodiments;

FIG. 4 depicts an illustrative flow diagram for displaying a state of mind via a real-time interactive digital embodiment of a patient in accordance with one or more example embodiments;

FIG. 5 depicts an illustrative real-time interactive digital embodiment of a patient in accordance with one or more example embodiments;

FIG. 6 depicts an illustrative view for displaying trends and comparisons via a real-time interactive digital embodiment of a patient in accordance with one or more example embodiments;

FIG. 7 depicts an illustrative view for displaying temporal information via real-time interactive digital embodiments in accordance with one or more example embodiments;

FIG. 8 depicts another illustrative view for displaying temporal information via real-time interactive digital embodiments in accordance with one or more example embodiments;

FIG. 9 depicts an illustrative view for displaying prescription information via real-time interactive digital embodiments in accordance with one or more example embodiments;

FIG. 10 depicts an illustrative frontal view for a real-time interactive digital embodiment of a patient in accordance with one or more example embodiments;

FIG. 11 depicts an illustrative dorsal view for a real-time interactive digital embodiment of a patient in accordance with one or more example embodiments;

FIG. 12 depicts an illustrative flow diagram for monitoring health attributes via a real-time interactive digital embodiment of a patient in accordance with one or more example embodiments; and

FIG. 13 depicts another illustrative flow diagram for monitoring health attributes via a real-time interactive digital embodiment of a patient in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. It is to be understood that other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired, or wireless, and that the specification is not intended to be limiting in this respect.

As described herein, various aspects of the system disclosed provide summarized and time stamped data of a patient's medical information as an interactive visual embodiment of the patient. The visual embodiment may be viewed by the patients and the doctors on their smartphones or computer screens, and inspected to review historical and current information about the patient. This may significantly reduce the physician's time, and may provide reassurances that relevant information will be available to the doctor—thereby improving decision making and patient experience.

Generally, during doctor-patient interactions, it may be significant for a physician to review a history of the patient's medical history. For example, it may be useful for a physician to know of medications that a patient has taken or is currently taking, a surgical history of the patient, a list of ailments, trends in the patient's medical history, a mental state of the patient, and so forth. As described herein, such information may not be available in one place, or may be available in paper documents in various formats, and so forth. In many instances, a physician may have to rely on a patient's account of the medical history, which may be incomplete, inaccurate, and/or inconsistent. In some instances, the physician may not be able to obtain medical history related to aspects of the patient's medical history that may be outside the practice area of the physician.

However, even if such information were made readily available in a convenient format, the physician may not be able to scan through such information, analyze the data, formulate treatment strategies, and determine the treatment. This is further exacerbated by a short duration for a doctor-patient interaction. Accordingly, it may be highly significant for a physician to have the patient's data available in a digital format, structured temporally, and presented in a succinct manner for ease of review. For example, the physician may select a date (or a date range) and review the patient's state of health during the selected time period. Furthermore, it may be highly significant for a physician to have a summary of salient features of the patient's medical history, along with snapshots of relevant aspects of the medical history, alerts associated with treatments and/or medications for the physician to check, and also recommended treatment strategies based on real-time analysis of large amounts of medical data, research data, drug related data, and so forth.

Generally, a doctor-patient interaction takes place when the patient is physically seen by the physician. However, it may be very beneficial for a physician to access a real-time digital embodiment of the patient and be able to track the patient's health over time, at any given time and even at a remote location without the patient being physically present in front of the physician. This may vastly improve a delivery of medical services, optimize resources, minimize human errors due to a lack of information and/or a lack of intelligent data and real-time analysis of the data. For example, a patient's medical data and historical trends may be compared to millions of records to determine optimal medical practices, minimize conflicting strategies, minimize drug interactions, and so forth. Accordingly, aspects of this disclosure provide effective, efficient, scalable, fast, reliable, and convenient technical solutions that address and overcome the technical problems associated with providing physicians and patients real-time, intelligent medical information and services. As described herein, a patient's medical history may be analyzed to provide personalized insights to patients and physicians, provide summaries and trends, provide medical alerts and notifications, and enable the physician to make medical determinations in a timely and reliable manner.

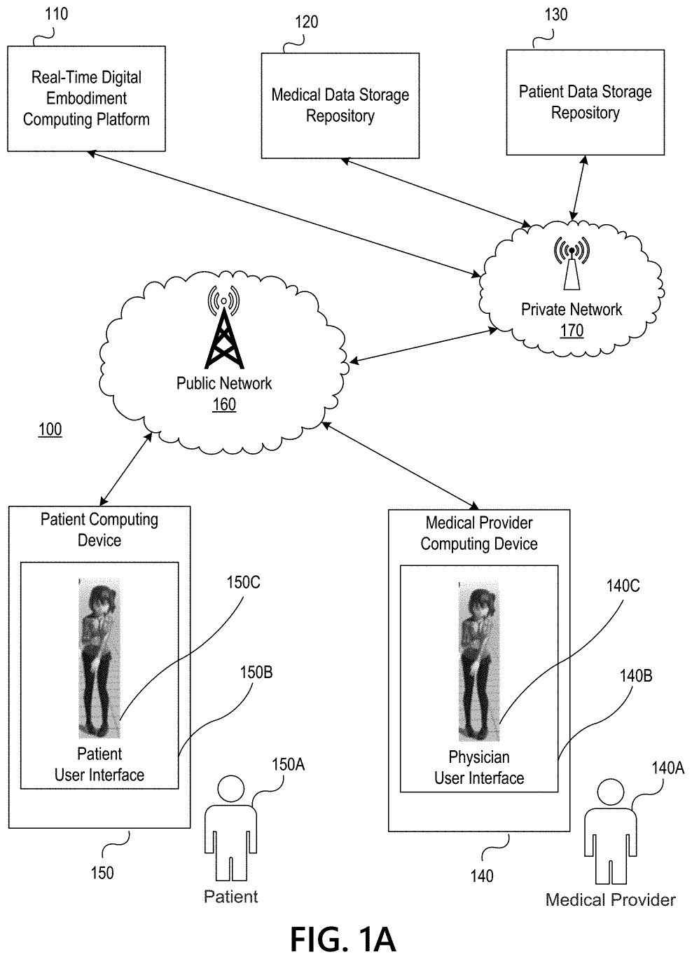

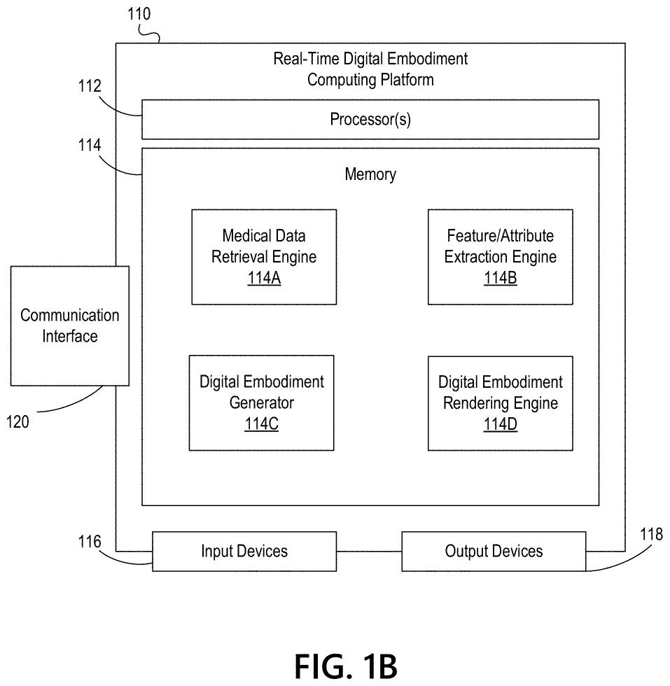

FIGS. 1A and 1B depict an illustrative computing environment for a real-time interactive digital embodiment of a patient in accordance with one or more example embodiments. Referring to FIG. 1A, computing environment 100 may include one or more computer systems. The term “system” may be used to refer to a single computing device or multiple computing devices that communicate with each other (e.g. via a network) and operate together to provide a unified service. For example, computing environment 100 may include real-time digital embodiment computing platform 110, medical data storage repository 120, patient data storage repository 130, medical provider computing device 140, and patient computing device 150.

In some embodiments, real-time digital embodiment computing platform 110 may include one or more computing devices configured to perform one or more of the functions described herein. For example, real-time digital embodiment computing platform 110 may include one or more computers (e.g., laptop computers, desktop computers, servers, server blades, or the like) and/or other computer components (e.g., processors, memories, communication interfaces).

Medical data storage repository 120 and patient data storage repository 130 may include one or more computing devices and/or other computer components (e.g., processors, memories, communication interfaces). In addition, and as illustrated in greater detail below, medical data storage repository 120 and patient data storage repository 130 may be configured to store and/or otherwise maintain medical data and patient data, including access controls to network devices and/or other resources hosted, executed, and/or otherwise provided by medical data storage repository 120. In addition, medical data storage repository 120 and patient data storage repository 130 may be configured to manage, host, execute, and/or otherwise provide one or more applications that perform the functions described herein. For example, medical data storage repository 120 and patient data storage repository 130 may be configured to manage, host, execute, and/or otherwise provide a computing platform that collects medical and/or patient data in unstructured format, converts such data into a structured format, indexes the data, and/or stores the data. In some embodiments, medical data storage repository 120 and patient data storage repository 130 may be configured to apply appropriate access controls and/or implement security measures to protect privacy and confidentiality of the data. As another example, medical data storage repository 120 and patient data storage repository 130 may be configured to store and/or otherwise maintain information associated with security profiles for applications (e.g., medical provider computing device 140, patient computing device 150). As another example, medical data storage repository 120 and patient data storage repository 130 may be configured to store and/or otherwise maintain data privacy classifications for information (e.g., personally identifiable information (PII), personal health information (PHI)). Additionally, or alternatively, real-time digital embodiment computing platform 110 may load data from medical data storage repository 120 and/or patient data storage repository 130, manipulate and/or otherwise process such data, and return modified data and/or other data to medical data storage repository 120 and/or patient data storage repository 130 and/or to other computer systems included in computing environment 100.

Medical provider computing device 140 and patient computing device 150 may be one or more computers (e.g., laptop computers, desktop computers, servers, server blades, or the like) and/or other computer components (e.g., processors, memories, communication interfaces). For example, medical provider computing device 140 may be a mobile device operated by a medical provider 140A. Also, for example, patient computing device 150 may be a mobile device operated by a patient 150A. In some aspects, medical provider computing device 140 may include a graphical user interface 140B to display a first digital embodiment 140C of a patient (e.g., patient 150A) to medical provider 140A. As will be described herein, the digital embodiment displayed to medical provider 140A may be configured to have appropriate restrictions on what data and/or information to display. In some instances, such restrictions may be controlled via access controls (e.g., based on a level of hierarchy and/or data access privileges for medical provider 140A). Also, for example, patient computing device 150 may include a graphical user interface 150B to display a second digital embodiment 150C of patient 150A to patient 150A.

As will be described herein, first digital embodiment 140C and second digital embodiment 150C may be different from one another. For example, first digital embodiment 140C may display information and/or data that patient 150A may not have access to. Also, for example, first digital embodiment 140C may display information and/or data that incorporates data from several patients that patient 150A may not have access to. As another example, first digital embodiment 140C may display information such as a cancerous growth, an amputated leg, and so forth to medical provider 140A, but not to patient 150A. Likewise, second digital embodiment 150C may be personalized by patient 150A to display personal information (e.g., personal health journal, personal exercise data, personal diet data, a daily mood analysis, calorie intake, beverage intake, an amount or time of sleep and so forth). In many instances, patient 150A may not share such data with medical provider 140A. In some embodiments, first digital embodiment 140C and second digital embodiment 150C may be identical, but may provide different information to each of medical provider 140A and patient 150A.

Computing environment 100 also may include one or more networks, which may interconnect one or more of real-time digital embodiment computing platform 110, medical data storage repository 120, patient data storage repository 130, medical provider computing device 140, and patient computing device 150. For example, computing environment 100 may include private network 170 (which may interconnect, for example, real-time digital embodiment computing platform 110, medical data storage repository 120, and patient data storage repository 130), and public network 160 (which may interconnect, for example, medical provider computing device 140, and patient computing device 150 with private network 170 and/or one or more other systems, public networks, sub-networks, and/or the like). For example, public network 160 may interconnect medical provider computing device 140 and/or patient computing device 150 with real-time digital embodiment computing platform 110, medical data storage repository 120, and patient data storage repository 130 via private network 170.

In one or more arrangements, real-time digital embodiment computing platform 110, medical data storage repository 120, patient data storage repository 130, medical provider computing device 140, and patient computing device 150, and/or the other systems included in computing environment 100 may be any type of computing device capable of communicating with a user interface, receiving input via the user interface, and communicating the received input to one or more other computing devices. For example, real-time digital embodiment computing platform 110, medical data storage repository 120, patient data storage repository 130, medical provider computing device 140, and patient computing device 150, and/or the other systems included in computing environment 100 may, in some instances, be and/or include server computers, desktop computers, laptop computers, tablet computers, smart phones, or the like that may include one or more processors, memories, communication interfaces, storage devices, and/or other components.

Referring to FIG. 1B, real-time digital embodiment computing platform 110 may include one or more processors 112, memory 114, input devices 116, output devices 118, and communication interface 120. A data bus may interconnect processor 112, memory 114, input devices 116, output devices 118, and communication interface 120. Communication interface 120 may be a network interface configured to support communication between real-time digital embodiment computing platform 110 and one or more networks (e.g., public network, private network, a local network, or the like). Memory 114 may include one or more program modules having instructions that when executed by processor 111 cause real-time digital embodiment computing platform 110 to perform one or more functions described herein and/or one or more databases that may store and/or otherwise maintain information which may be used by such program modules and/or processor 111. In some instances, the one or more program modules and/or databases may be stored by and/or maintained in different memory units of real-time digital embodiment computing platform 110 and/or by different computing devices that may form and/or otherwise make up real-time digital embodiment computing platform 110.

Input device 116, may include devices such as a microphone, keypad, keyboard, touchscreen, and/or stylus through which a user (e.g., medical provider 140A, patient 150A) may provide input data. An I/O module may also be configured to be connected to an output device 118 (e.g. a display device), such as a monitor, touchscreen, etc., and may include a graphics card. The display device and input device may be separate elements from the real-time digital embodiment computing platform 110; however, they may be within the same structure. In some embodiments, input device 116 may be operated by a patient to interact with real-time digital embodiment computing platform 110, including providing information about health attributes, physical attributes, emotional attributes, mental state, and so forth. Medical providers may use input device 116 to make updates such a medication related data, medical reports, diagnoses, and so forth.

For example, memory 114 may have, store, and/or include medical data retrieval engine 114A, feature/attribute extraction engine 114B, digital embodiment generator 114C, and digital embodiment rendering engine 114D. Medical data retrieval engine 114A engine may have instructions that direct and/or cause real-time digital embodiment computing platform 110 to retrieve, via the communication interface and from a medical repository, an electronic health record associated with a patient.

Feature/attribute extraction engine 114B may have instructions that direct and/or cause real-time digital embodiment computing platform 110 to extract, from the electronic health record, data indicative of: a plurality of patient features indicative of one or more of a physical feature or a mental state associated with the patient, and a plurality of health attributes of the patient. Digital embodiment generator 114C may have instructions that direct and/or cause real-time digital embodiment computing platform 110 to configure a digital embodiment of the patient to: display the plurality of patient features, and display information associated with the plurality of health attributes. Digital embodiment rendering engine 114D may have instructions that direct and/or cause real-time digital embodiment computing platform 110 to render, via a graphical user interface of a computing device, the digital embodiment of the patient. Other embodiments of this aspect include corresponding computer systems, apparatus, and computer programs recorded on one or more computer storage devices, each configured to perform the actions of the methods.

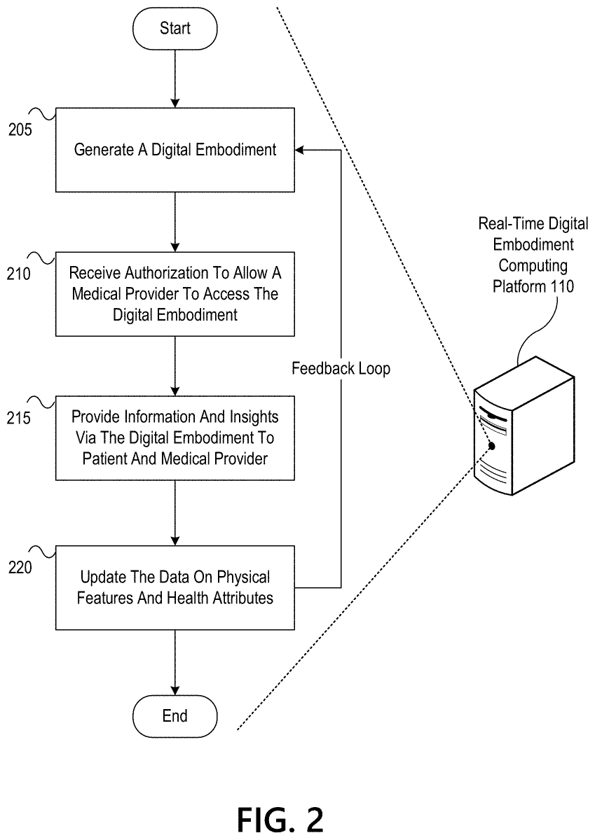

FIG. 2 depicts an illustrative flow diagram for a real-time interactive digital embodiment of a patient in accordance with one or more example embodiments. In some examples, such an illustrative flow diagram may be implemented by a system such as, for example, real-time digital embodiment computing platform 110 of FIG. 1. Referring to FIG. 2, at step 205, real-time digital embodiment computing platform 110 may generate a digital embodiment. At step 210 real-time digital embodiment computing platform 110 may receive authorization to allow a medical provider to access the digital embodiment. The term “medical provider,” as used herein, may generally refer to any person, facility, institution, service, and so forth that may provide medical related service to an individual. For example, the medical provider may be a physician, a nurse, a medical technician, a pharmacy, a laboratory, a diagnostics center, an investigations laboratory, an insurance provider, a hospital, a patient caregiver, an elder care center, and so forth.

In some embodiments, a patient may determine to provide access to a medical provider. Accordingly, the patient may select the medical provider, and indicate, via the graphical user interface, that the medical provider may access the digital embodiment of the patient. Real-time digital embodiment computing platform 110 may receive the indication, and provide a copy of the patient's digital embodiment to the medical provider. As described herein, the patient may choose a type of data that the patient may want to share with the medical provider.

In some embodiments, the patient may share the digital embodiment by providing the patient mobile device with a display of the digital embodiment. In some embodiments, a patient consent framework may be adopted (based on rules of a particular jurisdictional authority), and real-time digital embodiment computing platform 110 may configure a consent protocol compliant with the rules of the particular jurisdictional authority. In some embodiments, a consent may be automatic, for example, if a physician has discharged a patient from a hospital, and/or a patient is under active care of the physician. However, the consent protocol may be triggered if the patient seeks a second opinion from another physician.

At step 215, real-time digital embodiment computing platform 110 may provide information and insights, via the digital embodiment, to the patient and the medical provider. As described herein, different information and/or insights may be provided to the patient and the medical provider. For example, the patient's digital embodiment may provide insights and information pertaining to the patient's personal habits. However, the medical provider's digital embodiment may provide insights and information pertaining to the patient's medical reports, diagnoses, general trends for similar patients, and so forth.

At step 215, real-time digital embodiment computing platform 110 may update the data related to patient features and/or health attributes. For example, as the patient's height, weight, age, and so forth changes, real-time digital embodiment computing platform 110 may update the data. Also, with each doctor-patient interaction, real-time digital embodiment computing platform 110 may update the data. In some embodiments, the method may return to step 205 to update the digital embodiment based on the updated data.

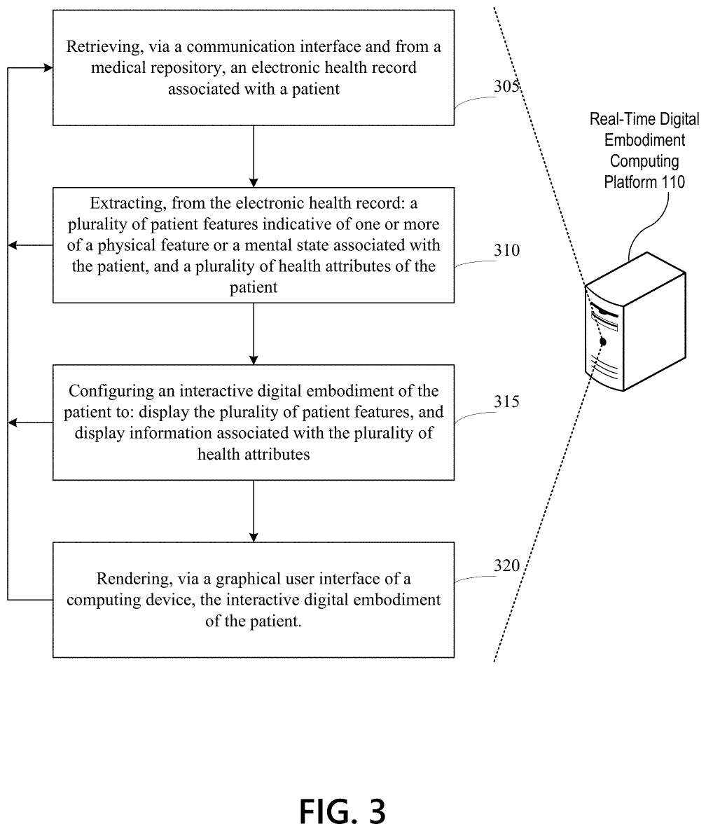

FIG. 3 depicts another illustrative flow diagram for a real-time interactive digital embodiment of a patient in accordance with one or more example embodiments. In some examples, such an illustrative flow diagram may be implemented by a system such as, for example, real-time digital embodiment computing platform 110 of FIG. 1. Referring to FIG. 3, at step 305, real-time digital embodiment computing platform 110 may retrieve, via the communication interface and from a medical repository, an electronic health record associated with a patient.

For example, real-time digital embodiment computing platform 110 may retrieve the electronic health record from medical data storage repository 120. An electronic health record may be a record of a patient's medical data, including, for example, prescriptions, procedures, investigations, and/or diagnoses. The medical data storage repository 120 may store medical data associated with the patient. For example, a patient may be prescribed a medication, and the patient may upload the prescription to medical data storage repository 120. As another example, the patient may undergo a medical procedure and a medical provider performing the medical procedure may upload information related to the medical procedure. Also, for example, a patient may have radiological tests performed on them, and a medical provider performing the radiological tests may upload information related to the tests. For example, x-ray and/or MM images may be stored in medical data storage repository 120. Accordingly, real-time digital embodiment computing platform 110 may retrieve such information from medical data storage repository 120.

At step 310, real-time digital embodiment computing platform 110 may extract, from the electronic health record, data indicative of: a plurality of patient features indicative of one or more of a physical feature or a mental state associated with the patient, and a plurality of health attributes of the patient. In some embodiments, real-time digital embodiment computing platform 110 may return to step 305 and retrieve another electronic health record. In some embodiments, the physical feature may include one or more of height, weight, skin complexion, color of eyes, hair color, hair style, a body mass index (BMI), gender, eye movement, voice, gait, items of clothing, clothing accessories (shoes, bracelets, anklets, earrings, handbags, etc.), and facial expression. In some embodiments, the mental state may include one or more of happy, sad, relaxed, depressed, excited, and so forth. Generally, the patient may customize their own digital embodiment. Also, for example, the plurality of health attributes of the patient may include attributes related to a health of various organs, medical diagnoses, and so forth.

At step 315, real-time digital embodiment computing platform 110 may configure a digital embodiment of the patient to: display the plurality of patient features, and display information associated with the plurality of health attributes. In some embodiments, real-time digital embodiment computing platform 110 may return to step 305 and retrieve another electronic health record. In some embodiments, real-time digital embodiment computing platform 110 may return to step 310 to extract additional attributes.

In some embodiments, real-time digital embodiment computing platform 110 may detect an interaction of the patient with a medical provider. For example, a patient may visit a physician for a physical examination, and real-time digital embodiment computing platform 110 may detect the interaction of the patient with the physician. Accordingly, real-time digital embodiment computing platform 110 may apply a timestamp to the digital embodiment of the patient, where the timestamp is indicative of the time of the interaction.

As another example, a patient may visit a medical imaging service provider for an x-ray, and real-time digital embodiment computing platform 110 may detect the interaction of the patient with the medical imaging service provider. Accordingly, real-time digital embodiment computing platform 110 may apply a timestamp to the digital embodiment of the patient, where the timestamp is indicative of the time of the interaction.

In some embodiments, real-time digital embodiment computing platform 110 may configure, for each interaction of the patient with the medical provider, a temporal version of the digital embodiment, where the temporal version is indicative of the electronic health record at the time of the interaction. For example, a patient may visit a medical imaging service provider for an x-ray, and real-time digital embodiment computing platform 110 may configure a temporal version of the digital embodiment, where the temporal version is indicative of a record of the medical images captured at the time of the interaction.

In some embodiments, the information associated with the particular health attribute may be located in a hierarchical level of a hierarchical structure of medical information. For example, the information may be associated with a tree structure. For example, a tree structure may correspond to information associated with a coding system LOINC. The information in LOINC is arranged in 25 chapters (e.g., hematology, urine analysis, microbiology, radiology, etc). For example, under the chapter for hematology, there may be further sub-topics such as for hemoglobin count, red blood cells (RBC) count, white blood cell (WBC) count, and so forth.

Accordingly, at a highest level, the physician may be able to view one or more of the 25 topics (e.g., hematology) highlighted for review. In some embodiments, real-time digital embodiment computing platform 110 may receive an indication that the physician has selected any one of these 25 topics (say hematology), then the information (e.g., hemoglobin count, RBC count, WBC count, etc.) from a second level of the hierarchical structure may be provided. Accordingly, the physician may view results for hemoglobin.

As another example, a chapter in LOINC corresponding to radiology, may include further sub-topics. Accordingly, at a highest level, real-time digital embodiment computing platform 110 may display radiology readings that the patient medical record shows, instead of displaying the theoretical tree. For example, a patient may have a chest X-ray (CXR) and an ultrasound, and radiology data may be displayed as green. Accordingly, the physician may immediately recognize that radiology data is normal and that there is no need to read the actual data or review the data itself. However, ultrasound data may not be normal, and radiology data may be displayed as orange or red, and this may provide an indication to the physician that further review may be required. In some examples, the physician may zoom in to a second level of the hierarchical information. At the second level, the CXR may be displayed as green whereas the ultrasound may be displayed as orange or red. Accordingly, the physician may recognize that the CXR is normal and may not require further review. However, the physician may recognize that the ultrasound may not be normal, and may require further review. In some embodiments, the physician may select the ultrasound data for more information. Subsequently, real-time digital embodiment computing platform 110 may display the ultrasound information via a text box, and may make the ultrasound report available for display.

In some embodiments, real-time digital embodiment computing platform 110 may detect a change in the electronic health record. Subsequently, real-time digital embodiment computing platform 110 may update, based on the detected change, the rendering of the digital embodiment. For example, real-time digital embodiment computing platform 110 may detect a change in hemoglobin count, and the hematology data point for the digital embodiment may be highlighted with a color red. Such color coding and/or hierarchical presentation of information may be a significant technological advancement. In the past, with paper records, the physician may have missed a report that may have impacted a treatment and/or diagnosis. However, with a presentation of digital data (e.g., hierarchical data, color coded information), the doctor may view the reports, and it may be difficult to miss adverse reports, as they are now highlighted.

In some embodiments, real-time digital embodiment computing platform 110 may detect a change based on a comparison of patient data and a normal range for such data. For example, the patient data may be in numerical format, and a normal range for such data may be a numerical range. Accordingly, real-time digital embodiment computing platform 110 may compare the patient data with the range, and determine whether the patient data is with the range, or outside the range. Also, for example, a deviation from the range may be determined to provide a degree of abnormality. In some embodiments, real-time digital embodiment computing platform 110 may perform predictive analysis based on historical data and population data to generate trend lines, and predict upcoming adverse medical events for the patient. A confidence level may be associated with such predictions based on statistical predictive models, and/or reliability models. In some embodiments, real-time digital embodiment computing platform 110 may identify patients similar to a patient, determine types of treatments that were advised, and outcomes of such treatments. Such analysis may further inform the trends, prediction, recommendations, and other actions as described herein.

As described herein, a special purpose computer may be configured to perform the operations. Generally, physicians are faced with (1) an information overload, (2) a short time for to review patient data, (3) access to limited patient data, and/or (4) a short window of time to examine a patient. Accordingly, physicians have to balance time with a depth of analysis of the information. In many instances, striking such a balance may come at the cost of an adverse effect on patient diagnosis and/or treatment. Also, for example, a physician may not have access to all data points of the patient, data related to similar patients, access to trends, and so forth. Accordingly, the digital embodiment may represent the data in a summarized digital format that enables the physician to have a real-time access to comprehensive medical history, real-time access to summarized content highlighting adverse reports with an ability to zoom in to review details, real-time access to Ix, Rx, Px etc. Also, for example, and these features may be available temporally for any doctor-patient interaction event. The information overload aspect may be mitigated by the hierarchical tree structure of the information (so if a chapter is green, then all child nodes in that chapter are also green and the physician need not review the entire chapter contents; similarly, if a chapter is red, the physician may be able to view the information for sub-topics for the specific chapter, without having to review all the reports). Also, for example, a short time to review information may be mitigated by the summarized content/report. Also, for example, access to limited patient data may be mitigated by the comprehensive nature of the medical data. As another example, a short window of time to examine a patient may be mitigated by the availability of the patient's digital embodiment (possibly updated with recent information), without the patient being physically present. Also, for example, comparisons with other patients for similar ailments etc. may be made available, and availability of data with regards to patient response to prior procedures, both for individual patients, and for a class of patients may be provided. Accordingly, the special purpose computing platform described herein solves a number of problems in a technological field of medical treatment.

In some instances, a patient may be examined by several physicians. At present, each physician may have limited visibility into how the other physician may be treating the patient. By bringing together pharmaceutical information into one digital embodiment, integrated care may be enabled. For example, a cardiologist may view what a neurosurgeon may be prescribing for epilepsy, an orthopedic surgeon may view what a cardiologist may be prescribing as a blood thinner, and so forth.

In some embodiments, real-time digital embodiment computing platform 110 may extract the plurality of patient features from a visual image or a video of the patient. For example, a patient may upload a photograph, and real-time digital embodiment computing platform 110 may perform image analysis to extract physical features, and/or a facial expression indicating a mental state, from the photograph. Also, for example, a patient may upload a video, and real-time digital embodiment computing platform 110 may perform video analysis, and/or facial recognition techniques, to extract physical features, and/or a facial expression indicating a mental state from the video. In some embodiments, real-time digital embodiment computing platform 110 may extract features related to a gait, a posture, and so forth. In some embodiments, real-time digital embodiment computing platform 110 may extract voice features of the patient from an analysis of the video. Subsequently, real-time digital embodiment computing platform 110 may configure the digital embodiment based on such extracted features. For example, real-time digital embodiment computing platform 110 may configure the digital embodiment to mimic facial expressions, mimic a gait, replicate a posture, hair color, color of eyes, mimic a voice, depict a mental state, and so forth.

In some embodiments, real-time digital embodiment computing platform 110 may animate a face of the digital embodiment to display one or more facial expressions. For example, one or more states of mind may be associated with facial expressions. In some embodiments, the states of mind may include, for example, “Excited,” “Astonished,” “Delighted,” “Happy,” “Pleased,” “Content,” “Serene,” and so forth.

In some embodiments, real-time digital embodiment computing platform 110 may identify, for each facial expression, a collection of facial muscles associated with the facial expression. For example, for each state of mind, a collection of facial muscles may be detected that are associated with each state of mind.

Subsequently, real-time digital embodiment computing platform 110 may associate, for the collection of facial muscles, a set of rules that mimic the facial expression on the face of the digital embodiment. In some embodiments, real-time digital embodiment computing platform 110 may determine, for each collection of facial muscles, the rules that may be utilized to mimic that state of mind. For example, a collection of facial muscles that cause a person to smile, may be associated with a set of rules that cause a smile to appear on a face of the digital embodiment.

In some embodiments, real-time digital embodiment computing platform 110 may receive information related to a state of mind for the patient. For example, a person may use their mobile computing device to indicate a state of mind. Subsequently, real-time digital embodiment computing platform 110 may associate a facial expression with the state of mind. Based on the state of mind, real-time digital embodiment computing platform 110 may associate a facial expression. Then, real-time digital embodiment computing platform 110 may configure a face of the digital embodiment for the patient to display the associated facial expression.

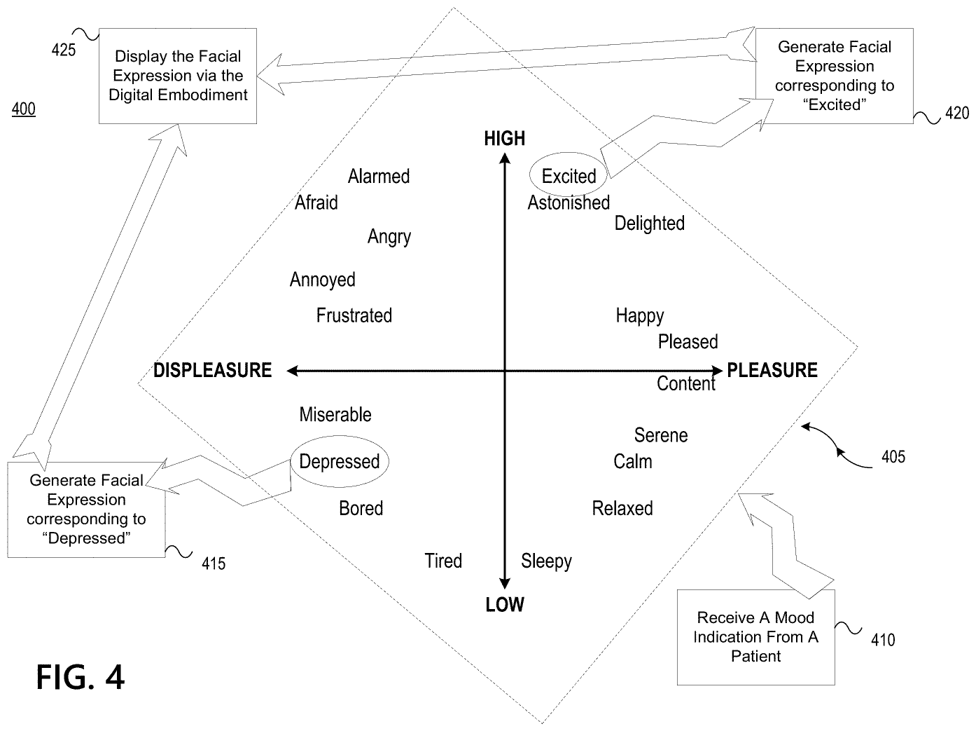

FIG. 4 depicts an illustrative flow diagram for displaying a state of mind via a real-time interactive digital embodiment of a patient in accordance with one or more example embodiments. For example, facial muscles may be used to represent a state of mind of the patient (based on a mood framework). As illustrated, a mood framework 405 may be represented on rectangular coordinates. The right portion of the horizontal axis may represent a degree of “Pleasure”, and the left portion of the horizontal axis may represent a degree of “Displeasure.” Similarly, top portion of the vertical axis may represent a degree of “High” emotive state, and the bottom portion of the vertical axis may represent a degree of “Low” emotive state. In some embodiments, any position in such a framework may be associated with a unique coordinate, and each coordinate may be associated with a unique score.

At step 410, real-time digital embodiment computing platform 110 may receive a mood indication from a patient. For example, the patient may indicate a mood of being “Depressed.” Accordingly, according to one or more aspects described herein, real-time digital embodiment computing platform 110 may, at step 415, generate a facial expression corresponding the mood of being “Depressed.” In some embodiments, at step 425, real-time digital embodiment computing platform 110 may display the facial expression via the digital embodiment. Accordingly, when a physician views the digital embodiment, the physician may see that the patient is depressed, even though the patient may be physically removed from the physician.

As another example, at step 410, real-time digital embodiment computing platform 110 may receive a mood indication from a patient. For example, the patient may indicate a mood of being “Excited.” Accordingly, according to one or more aspects described herein, real-time digital embodiment computing platform 110 may, at step 420, generate a facial expression corresponding the mood of being “Excited.” In some embodiments, at step 425, real-time digital embodiment computing platform 110 may display the facial expression via the digital embodiment. Accordingly, when a physician views the digital embodiment, the physician may see that the patient is excited, even though the patient may be physically removed from the physician.

Also, for example, states of mind, such as, for example, “relaxed” and/or “calm” may be represented via the digital embodiment. In some embodiments, a score may be assigned to each trait on a mood indicator, and one or more of such traits may be combined to cause complex movements of facial muscles. Such complex movements may be transformed to a depiction of complex emotional states via facial expressions on the digital embodiment.

In some embodiments, real-time digital embodiment computing platform 110 may associate, with the patient, a wellness score indicative of the patient's well-being. For example, a patient may indicate that a state of their well-being is “feeling fine,” and may be associated with a wellness score of “10/10”. As another example, a patient may indicate that a state of their well-being is “feeling ill” may be associated with a wellness score of “2/10”. In some embodiments, the patient may select the wellness score.

Subsequently, real-time digital embodiment computing platform 110 may associate, for the digital embodiment, a body posture with the wellness score. In some examples, the body posture may include, for example, an arm position, a leg position, a head position, and so forth. For example, different arm positions may be indicated via the digital embodiment, and these arm positions may indicate to the physician information about the state of well-being of the patient.

Real-time digital embodiment computing platform 110 may configure the body posture of the digital embodiment for the patient to display the wellness score. For example, when the body posture is an arm position, a raised arm position may be associated with a state of well-being corresponding to “feeling fine,” an arm position at 60° may correspond to a wellness score of “7/10,” a horizontal arm position may correspond to a wellness score of “5/10,” and a lowered arm position may correspond to a state of well-being corresponding to “feeling ill” and/or a wellness score of “2/10.”

In some embodiments, the wellness score may be received as an input from the patient. Generally, a patient may input their state of well-being, and such data may be timestamped and entered into the database.

In some embodiments, real-time digital embodiment computing platform 110 may associate, for the digital embodiment, a body posture with a temporal trend. For example, a patient may have myocardial infraction and their left ventricular injection infraction may be steadily decreasing, and liver function tests may be demonstrating a worsening trend. Such data may be displayed to a physician via an arm position of the digital embodiment. For example, real-time digital embodiment computing platform 110 may identify medical features that may be tracked and a health trend may be output based on the medical data, and represented as physical images. Medical features may include, for example, a kidney function test, blood urea, serum creatinine, liver function test (prothrombin time (PT/INR), activated Partial Thromboplastin Time (aPTT), albumin, bilirubin (direct and indirect), and others such as alkaline phosphate), heart function test (ECG, co-cardiograph), pulmonary function test, EPIv1 (exhalation volume within 1 second), neurological tests, and so forth.

In some embodiments, real-time digital embodiment computing platform 110 may associate, with each health attribute of the plurality of health attributes, an attribute score. In some embodiments, the wellness score may be an aggregate of attribute scores. In some embodiments, real-time digital embodiment computing platform 110 may determine, for each health attribute of the plurality of health attributes, a temporal trend. In some embodiments, real-time digital embodiment computing platform 110 may configure the body posture of the digital embodiment for the patient to display the temporal trend.

Attribute scores may be associated with each health attribute, and an aggregate score may be generated. For example, real-time digital embodiment computing platform 110 may determine if the aggregate score is increasing, and may configure the arm position to move up. Also, for example, real-time digital embodiment computing platform 110 may determine if the aggregate score is decreasing, and may configure the arm position to move down. As another example, real-time digital embodiment computing platform 110 may determine if the aggregate score does not show a perceptible change, and may configure the arm position to remain the same.

At step 320, real-time digital embodiment computing platform 110 may render, via a graphical user interface of a computing device, the digital embodiment of the patient. In some embodiments, real-time digital embodiment computing platform 110 may return to step 305 and retrieve another electronic health record. In some embodiments, real-time digital embodiment computing platform 110 may return to step 310 to extract additional attributes. In some embodiments, real-time digital embodiment computing platform 110 may return to step 315 to configure the digital embodiment. It may be noted that the above steps may not be performed in a strict sequence. For example, one or more of these steps may be performed simultaneously.

In some embodiments, sensitive health information may be displayed in a manner so as to minimize distress to the patient. For example, real-time digital embodiment computing platform 110 may not depict, in the digital embodiment, a bald head of a cancer patient undergoing chemotherapy. Also, for example, real-time digital embodiment computing platform 110 may not depict, in the digital embodiment, an amputated limb of a patient.

A physician may view a large number of digital embodiments associated with different patients. Accordingly, it may be useful for the physician to be able to distinguish between the different patients. In some embodiments, a physical resemblance to a patient (e.g., based on the plurality of patient features), and/or medical information (e.g., based on the plurality of health attributes) may help personalize the digital embodiment. This may enable a patient to be comfortable with the digital embodiment, and enable the physician to recognize the patient from the digital embodiment.

Generally, real-time digital embodiment computing platform 110 may configure and render the digital embodiment to function as an operating system between the medical provider and the patient. For example, a physician may utilize and review the patient information in a user-friendly manner. Also, for example, a patient may review their information, so that they may take better ownership of their health data, and exercise a greater degree of control over their health in general.

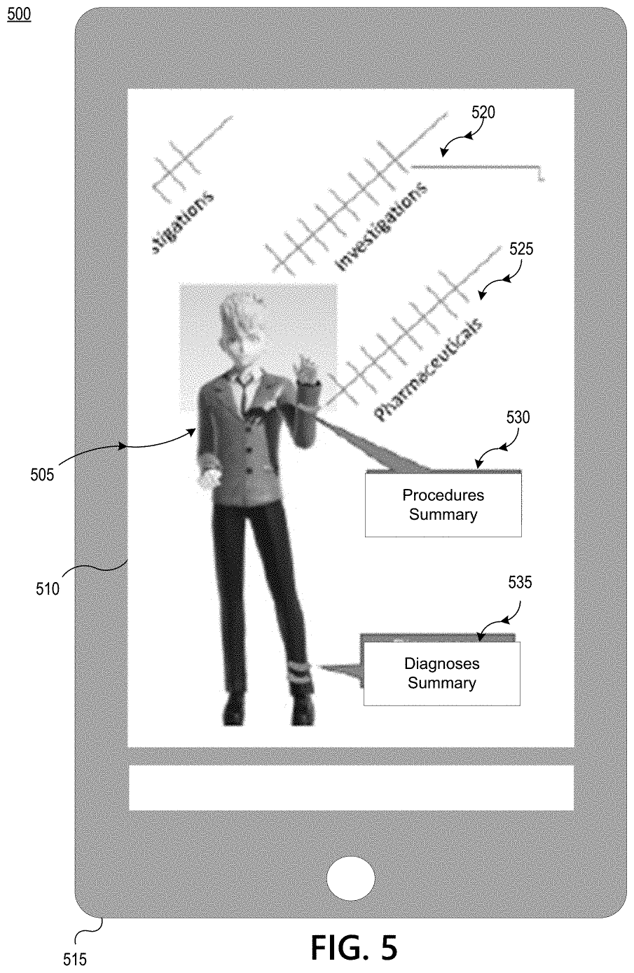

FIG. 5 depicts an illustrative view 500 for a real-time interactive digital embodiment of a patient in accordance with one or more example embodiments. In some embodiments, real-time digital embodiment computing platform 110 may provide the digital embodiment 505 via a graphical user interface 510 of a mobile device 515. As illustrated, digital embodiment 505 may be utilized to provide patient and/or medical data in a summarized digital format that enables a physician to have a real-time access to comprehensive medical history, real-time access to summarized content highlighting adverse reports with an ability to zoom in to review details, real-time access to Ix, Rx, Px etc. Generally, digital embodiment 505 may be configured to display information from four Health Information Categories that are medically relevant, i.e., Investigations [Ix], Prescriptions [Rx], Diagnosis [Dx] and Procedure [Px] may be represented by icons.

For example, Investigations 520 may provide information related to medical investigations performed on the patient. Such information may be temporal, hierarchical, and so forth, and may include data from several physicians that may have treated the patient. As another example, Pharmaceuticals 525 may provide information related to medications prescribed to the patient. As another example, Procedures Summary 530 may provide information related to procedures (e.g., surgical procedures) performed on the patient. Also, for example, Diagnosis Summary 535 may provide information related to medical diagnoses (e.g., inflamed liver, asthma, schizophrenia, congestive heart failure) for the patient.

In some embodiments, real-time digital embodiment computing platform 110 may detect, from the electronic health record, presence of a medical implant in the patient. The medical implant may be a device or a tissue. In some embodiments, the medical implant may be a prosthetic. In some embodiments, the medical implant may be a device utilized to deliver medication, manage, support, and/or monitor body parts. For example, the medical implant may be an Implantable Cardioverter Defibrillators (ICDs), an artificial hip, an artificial knee, coronary stents, ear tubes, a pacemaker for the heart, a breast implant, intra-uterine devices (IUDs), artificial eye lenses, and so forth. Also, for example, the medical implant may include screws, rods, and/or artificial discs for the vertebral column. As another example, the medical implant may include devices for traumatic bone fracture repair, such as, for example, metal screws, plates, pins, and rods. As another example, the medical implant may be a transplanted organ, such as a transplanted kidney, liver, and so forth.

Then, real-time digital embodiment computing platform 110 may determine, from the electronic health record, a physical location of the medical implant. For example, the medical implant may be an artificial knee, and real-time digital embodiment computing platform 110 may determine whether it is the left or the right knee. As another example, the medical implant may be breast implant, and real-time digital embodiment computing platform 110 may determine whether it is the left or the right breast. Also, for example, the medical implant may be a coronary stent, and real-time digital embodiment computing platform 110 may determine an artery, and a location of the stent in the artery.

Subsequently, real-time digital embodiment computing platform 110 may configure the interactive digital embodiment of the patient to display an indication of the medical implant at a location, on the digital embodiment, that corresponds to the physical location. For example, if the medical implant is an artificial knee on the left knee, real-time digital embodiment computing platform 110 may configure the interactive digital embodiment of the patient to display an indication of the artificial knee on the left knee of the digital embodiment. As another example, if the medical implant is an artificial disc that replaced the 7^{th} vertebra, real-time digital embodiment computing platform 110 may configure the interactive digital embodiment of the patient to display an indication of the artificial disc at the location of the 7^{th} vertebra of the digital embodiment.

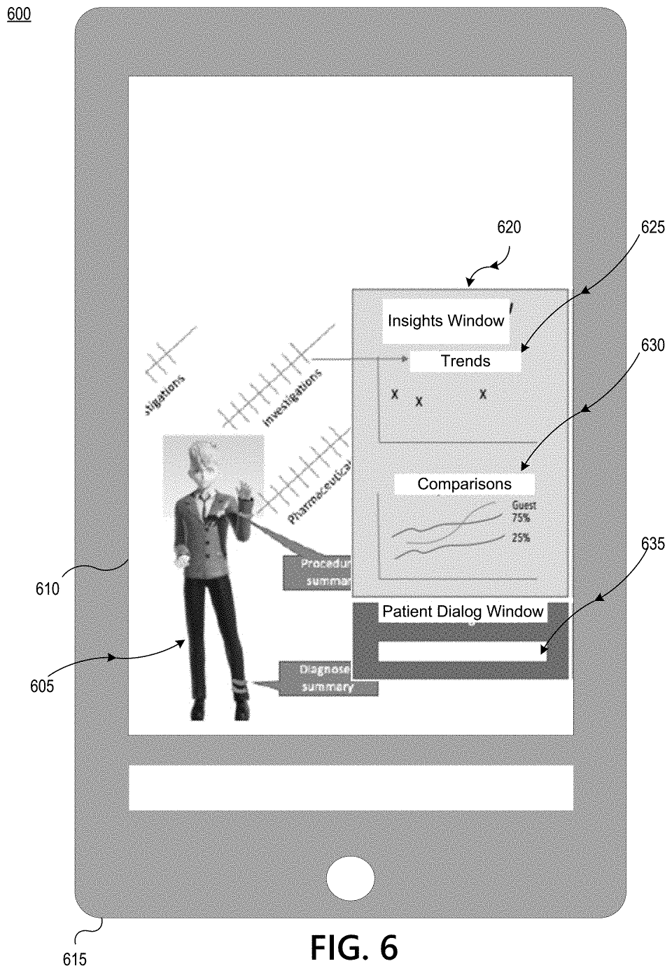

FIG. 6 depicts an illustrative view 600 for displaying trends and comparisons via a real-time interactive digital embodiment of a patient in accordance with one or more example embodiments. In some aspects, the features illustrated in FIG. 6 may be similar to those described with reference to FIG. 5. In some embodiments, real-time digital embodiment computing platform 110 may provide the digital embodiment 605 via a graphical user interface 610 of a mobile device 615. For example, a physician may have selected Procedures Summary 530 of FIG. 5. Accordingly, real-time digital embodiment computing platform 110 may generate a report 620 summarizing information related to procedures performed on the patient. In some aspects, report 620 may be presented as a pop-up window. In some instances, report 620 may include an insights window illustrating trends 625 indicative of trends associated with the patient data. For example, if several recent heart procedures have been performed, then real-time digital embodiment computing platform 110 may indicate such a trend. As another example, report 620 may include an insights window illustrating comparisons 630. Comparisons 630 may be a comparison of patient's data with data from similar patients. For example, comparisons 630 may provide comparative data based on age, race, gender, geographic location, income category, profession, insurance coverage, patients with similar ailments, and so forth. Patient Dialog Window 635 may be an interface for a medical provider to exchange data and information with a patient.

In some embodiments, real-time digital embodiment computing platform 110 may render, via the graphical user interface of the computing device, a plurality of temporal versions of the digital embodiment arranged in chronological order, where each temporal version of the plurality of temporal versions is associated with the time of the interaction. For example, the display may provide several versions of the same digital embodiment, arranged in line, with each version representing a specific time period. Upon a selection of a version via the graphical user interface, the selected version may display healthcare information from the time period associated with that version.

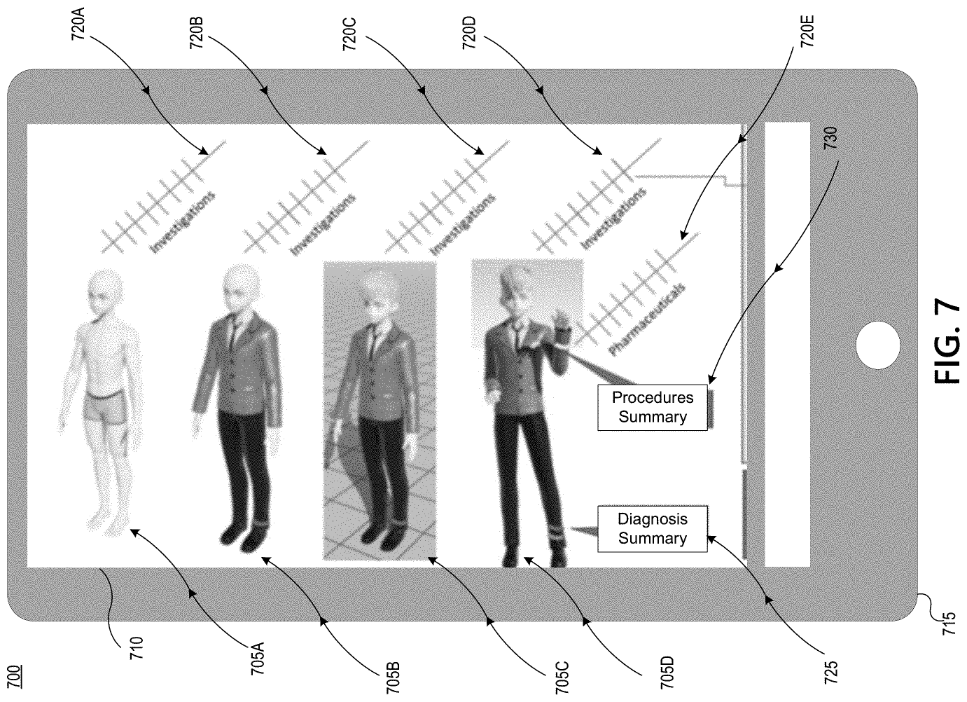

FIG. 7 depicts an illustrative view 700 for displaying temporal information via real-time interactive digital embodiments in accordance with one or more example embodiments. In some embodiments, real-time digital embodiment computing platform 110 may provide digital embodiments 705A-D via a graphical user interface 710 of a mobile device 715. In some aspects, each digital embodiment may be configured to display and/or provide one or more features as described with reference to FIG. 5 or FIG. 6.

In some embodiments, a first version of digital embodiment 705A may be associated with a first time, a second version of digital embodiment 705B may be associated with a second time, a third version of digital embodiment 705C may be associated with a third time, and a fourth version of digital embodiment 705D may be associated with a fourth time. In some embodiments, the first time may be associated with a first time of a doctor-patient interaction. In some embodiments, the second may be associated with a second time of a doctor-patient interaction, and so forth. Also, for example, the first version of digital embodiment 705A may be associated with a summary of Investigations 720A, the second version of digital embodiment 705B may be associated with a summary of Investigations 720B, the third version of digital embodiment 705C may be associated with a summary of Investigations 720C, and the fourth version of digital embodiment 705D may be associated with a summary of Investigations 720D. As indicated, the fourth version of digital embodiment 705D may be associated with a summary of Pharmaceuticals 720E, Diagnosis Summary 725, and Procedures Summary 730.

In some embodiments, real-time digital embodiment computing platform 110 may update, in real-time, the rendering of the digital embodiment, and/or a time stamp associated with the digital embodiment. This is a significant aspect of the technology as described herein. Real-time digital embodiment computing platform 110 may receive and/or process large volumes of data. Such data may be received from a large number of sources (medical databases, patients' mobile devices, physicians' mobile devices, hospital databases, pharmacies, and so forth). Real-time digital embodiment computing platform 110 may continually perform analyses on such data, identifying trends, extracting insights, and so forth. Based on such updates, real-time digital embodiment computing platform 110 may continually update the configuration and/or the rendering of digital embodiments. Accordingly, a patient and/or a physician may access digital embodiments that may represent updated data for Investigations, Procedures, Diagnoses, and/or Pharmaceuticals. Also, for example, a current state of a patient's well-being, state of mind, and so forth may be provided.

In some embodiments, the digital embodiment may be a three-dimensional rendering of the patient. For example, real-time digital embodiment computing platform 110 may render a three-dimensional version of the digital embodiment. Also, for example, real-time digital embodiment computing platform 110 may configure the display so that the digital embodiment may be rotated, animated, moved, and so forth. In some embodiments, real-time digital embodiment computing platform 110 may configure the digital embodiment to make gestures (e.g., hand gestures, facial gestures, and so forth). Also, for example, the digital embodiment may be configured to move around, jump around, and display fighter poses like a Ninj a. As another example, the digital embodiment may be configured to be viewable at different angles, and/or perspectives (top, bottom, front, back, side, and so forth). Also, for example, the digital embodiment may be configured to be viewable at different resolutions, and configured with zoom-in, and/or zoom-out features.

Generally, a three-dimensional rendering may enable a physician to examine a spine, hip joint, hemorrhoids, anal fissure, and so forth. In some embodiments, the physician may turn and position the digital embodiment. Such an interaction of the physician with the digital embodiment of the patient may be a virtual examination of the patient, analogous to a physical examination of the patient. A physician may typically use their hand, a stethoscope, or a hammer to physically examine a patient. In some embodiments, real-time digital embodiment computing platform 110 may represent the same information on the digital embodiment via a coloring scheme, reports, and so forth. Generally, information extracted by a physician from a real-time physical examination, may be obtained by interacting with the digital embodiment. Also, for example, as an advantage to a physical examination, real-time digital embodiment computing platform 110 may provide the physician with historical patient data and trends.

In some embodiments, real-time digital embodiment computing platform 110 may detect, via the graphical user interface, a user interaction indicative of a movement associated with the digital embodiment. For example, real-time digital embodiment computing platform 110 may detect a selection of an icon, an input, an indication of a zoom functionality, and so forth.

Subsequently, real-time digital embodiment computing platform 110 may cause the digital embodiment to perform the indicated movement. For example, a liver specialist may be able to view an angioplasty or a knee replacement, and in a snapshot, the liver specialist may have access to information about the patient. Real-time digital embodiment computing platform 110 may display this to a physician. The physician may be able to zoom in and view three stents. For example, a three-dimensional rendition of an MM may be provided at the region corresponding to a part of a body. Additional and/or alternative radiological information may also be provided. For example, iconic images of implants, 3D renderings of radiology data, and so forth may be displayed via the digital embodiment. In some embodiments, a physician and/or a patient may capture a photo, or a video, and/or other data (e.g., a sound of a heartbeat etc.), and may upload such data to the appropriate region of the digital embodiment.

In some embodiments, the computing device may be associated with the patient, and real-time digital embodiment computing platform 110 may perform the generating based on one or more of a sub-plurality of the plurality of patient features, and a sub-plurality of the plurality of health attributes. For example, a patient may not have access to all the data trends and/or analyses that are available to the physician. Accordingly, real-time digital embodiment computing platform 110 may generate the digital embodiment for the patient's view based on features and/or functionalities that are available to the patient. In some embodiments, real-time digital embodiment computing platform 110 may provide the generated digital embodiment to the computing device associated with the patient.

In some embodiments, the computing device may be associated with a medical professional with an access to the electronic health record of the patient, and real-time digital embodiment computing platform 110 may perform the generating based on a sub-plurality of the plurality of patient features. For example, a patient may not share personal aspects of the digital embodiment with the physician. Also, for example, a physician may be privy to trends, recommendations, analyses, and so forth that may not be accessible to the patient. Accordingly, real-time digital embodiment computing platform 110 may generate the digital embodiment for the physician's view based on features and/or functionalities that are available to the physician and/or v. In some embodiments, real-time digital embodiment computing platform 110 may provide the generated digital embodiment to the computing device associated with the medical professional.

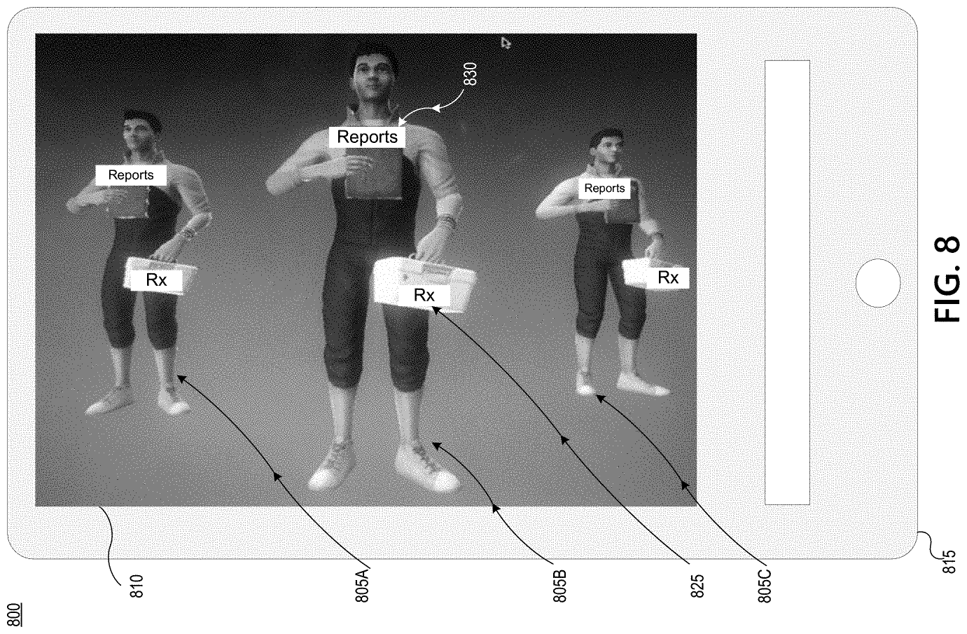

FIG. 8 depicts another illustrative view 800 for displaying temporal information via real-time interactive digital embodiments in accordance with one or more example embodiments. For example, there may have been three doctor-patient interactions in the last five years. Accordingly, real-time digital embodiment computing platform 110 may provide information at a high-level indicating procedures, conditions, and diagnoses that have been carried out, trends of blood tests, etc. In some embodiments, real-time digital embodiment computing platform 110 may provide digital embodiments 805A-C via a graphical user interface 810 of a mobile device 815. In some aspects, each digital embodiment may be configured to display and/or provide one or more features as described with reference to FIG. 5, FIG. 6, and/or FIG. 7.

In some embodiments, a first version of digital embodiment 805A may be associated with a first doctor-patient interaction, a second version of digital embodiment 705B may be associated with a second doctor-patient interaction, a third version of digital embodiment 705C may be associated with a third doctor-patient interaction. Also, for example, as illustrated, the second version of digital embodiment 805B may be associated with a summary of Reports 830, and a summary of pharmaceuticals Rx 820.

The term “zoom” as used herein may correspond to several types of “zoom” features. For example, a temporal zoom may be performed to focus on information from a specified time. As another example, an organ-level zoom may be performed to focus on information for a specific organ. Also, for example, an information level zoom may be performed to focus on a type of information. As another example, a hierarchical zoom may be performed to drill down into different levels of hierarchical information.

For example, a physician may select a digital embodiment specific to a time, (e.g., a temporal zoom-in), and real-time digital embodiment computing platform 110 may display the digital embodiment corresponding to the specific time. Also, for example, the digital embodiment corresponding to the specific time may be displayed with patient information from that time, with the features as described herein. In some embodiments, digital embodiments from different times may be displayed together. In some embodiments, a physician may select a time, and real-time digital embodiment computing platform 110 may cause the digital embodiment corresponding to the selected time to step forward, and walk to a foreground of the display screen. As the digital embodiment walks, additional interactive features (e.g., example, an arm position indicating health, facial expressions indicating mood, Rx, Ix, information as a tree, and so forth), may be displayed for the physician to interact with. In some embodiments, real-time digital embodiment computing platform 110 may cause digital embodiments representative of times other than the selected time, to fade in the background, and/or diminish in size.

Also, for example, the physician may choose an organ-level zoom-in to focus on more information about a particular organ, or an information level zoom-in for specific information (e.g., only look at medicines, diagnosis, past procedures, etc.).

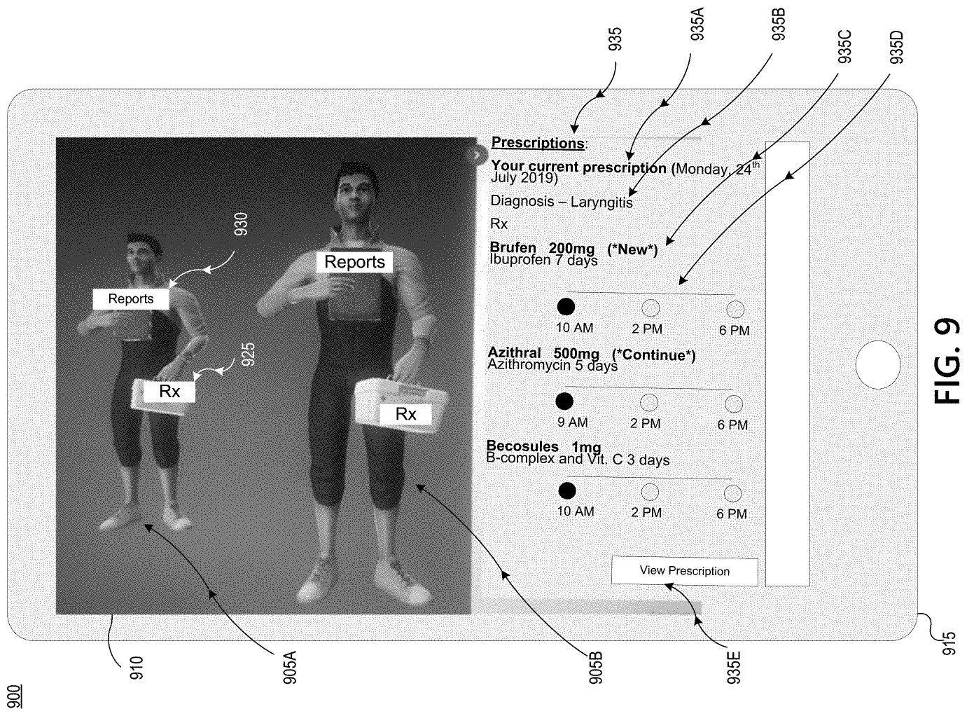

FIG. 9 depicts an illustrative view 900 for displaying prescription information via real-time interactive digital embodiments in accordance with one or more example embodiments. In some embodiments, real-time digital embodiment computing platform 110 may provide digital embodiments 905A and 905C via a graphical user interface 910 of a mobile device 915. In some aspects, each digital embodiment may be configured to display and/or provide one or more features as described with reference to FIG. 8.

In some embodiments, a first version of digital embodiment 905A may be associated with a first doctor-patient interaction, and a second version of digital embodiment 905B may be associated with a second doctor-patient interaction. Also, for example, as illustrated, the first version of digital embodiment 905A may be associated with a summary of Reports 930, and a summary of pharmaceuticals Rx 925.

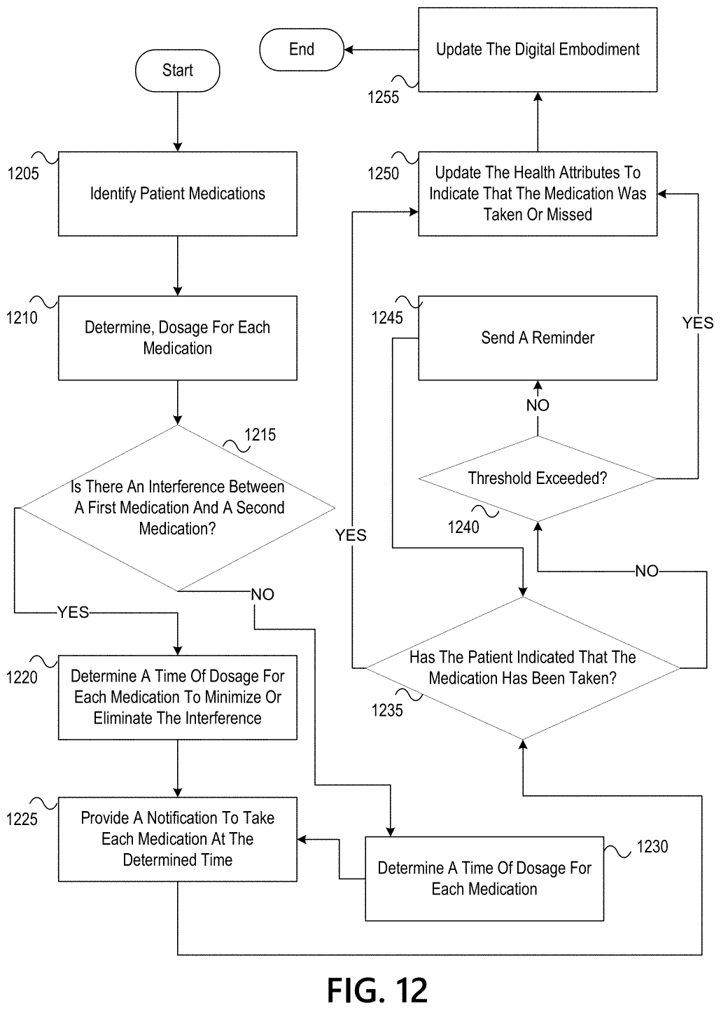

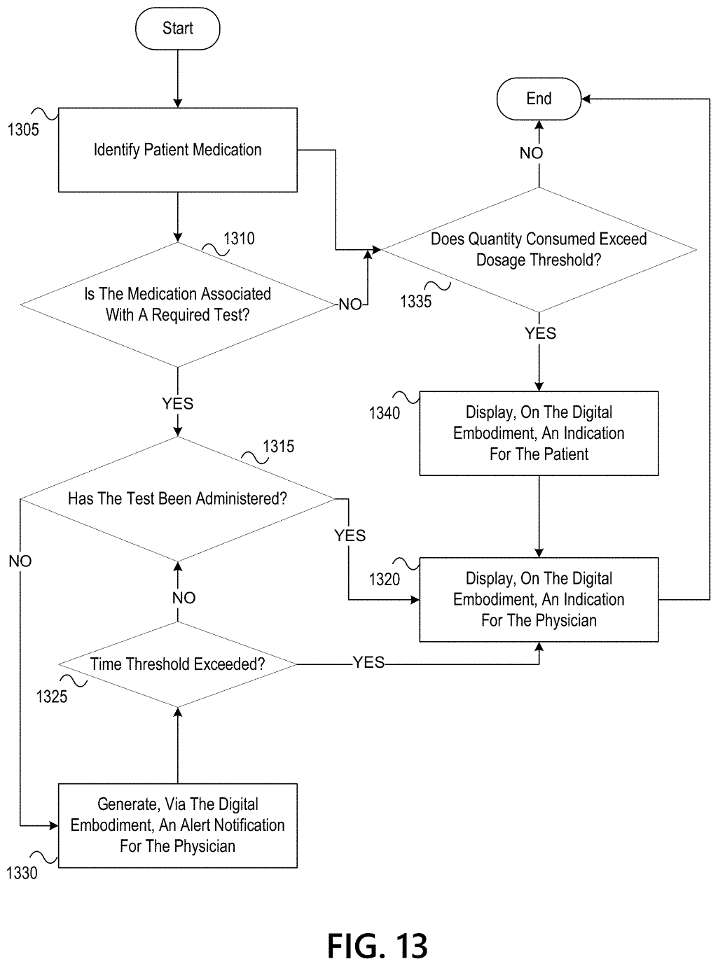

In some aspects, a patient may select pharmaceuticals Rx 925, and real-time digital embodiment computing platform 110 may detect such a selection, and may generate a Report on Prescriptions 935 summarizing information related to medications prescribed to the patient. For example, a report heading 935A may state, “Your current prescription (Monday, 24 Jul. 2019).” Also, for example, the Report on Prescriptions 935 may indicate a diagnosis 935B as “Laryngitis”. The Report on Prescriptions 935 may then list the medications. For example, a first medication 935C may be indicated as a “New” medication that has been prescribed recently. Also, for example, a dosage for the medication may be provided (e.g., 7 days), and a timeline 935D for taking the medication may be provided. For example, real-time digital embodiment computing platform 110 may indicate that the first dosage at 10 AM was taken (indicated by a filled-in circle), whereas a second dosage is to be taken at 2 PM, and a third dosage may be taken at 6 PM. As described with reference to FIG. 12, one or more reminders may be provided. Also, for example, as described with reference to FIG. 13, interferences between medications may be determined and the timeline 935D may be updated accordingly. As another example, as described with reference to FIG. 13, if a dosage level is exceeded, a notification may be provided. In some embodiments, a selectable tab 935E may be provided. Upon selection of tab 935E, the patient's prescription may be provided, and be made available for print, transmission to another physician, transmission to a pharmacy, and so forth.

In some embodiments, real-time digital embodiment computing platform 110 may identify, for a particular health attribute of the plurality of health attributes, a particular location on or around the digital embodiment corresponding to the particular health attribute. For example, for a health attribute associated with the heart, the particular location on the digital embodiment may correspond to a region of the heart. As another example, for a health attribute associated with the brain, the particular location on or around the digital embodiment may correspond to a region of the brain. Subsequently, real-time digital embodiment computing platform 110 may display the information associated with the particular health attribute at the particular location on or around the digital embodiment.

In some embodiments, real-time digital embodiment computing platform 110 may associate, with each organ of the patient and based on the electronic health record, a health score indicative of a health of the organ. Then, real-time digital embodiment computing platform 110 may associate, with each health score, a color scheme. For example, real-time digital embodiment computing platform 110 may segment a human body into regions for various organs, such as, for example, liver, heart, kidney, lung, brain, and so forth. Each region may be associated with a health score. For example, a condition of a healthy heart may be associated with a health score of “10/10”, or “healthy” and so forth. In some embodiments, the health score may be associated with a color scheme, such as, for example, a color “red” indicating a health score “bad,” a color “orange” indicating a health score “okay,” and a color “green” indicating a health score “good.”

In some embodiments, real-time digital embodiment computing platform 110 may display, for the region and based on the health score associated with the organ, a color from the color scheme. In some embodiments, such colors may be associated with the region corresponding to an organ. Subsequently, real-time digital embodiment computing platform 110 may determine, for each organ of the patient, a region of the digital embodiment associated with the organ. For example, different regions of the digital embodiment may be associated with one or more organs. In some embodiments, real-time digital embodiment computing platform 110 may display a color associated with a health score for the organ. For example, the patient's heart may be associated with a health score “good,” and real-time digital embodiment computing platform 110 may display a color “green” at the region of the digital embodiment associated with the heart. As another example, the patient's liver may be associated with a health score “bad,” and real-time digital embodiment computing platform 110 may display a color “red” at the region of the digital embodiment associated with the liver. Also, for example, the patient's kidney may be associated with a health score “okay,” and real-time digital embodiment computing platform 110 may display a color “orange” at the region of the digital embodiment associated with the kidney. Accordingly, physician may obtain a snapshot, and may detect that some areas have not been previously examined and may decide to do so. Also, the coloring scheme may enable a physician to make sure to review/analyze the regions colored “red” and/or “orange” so that concerns are not ignored and/or missed.

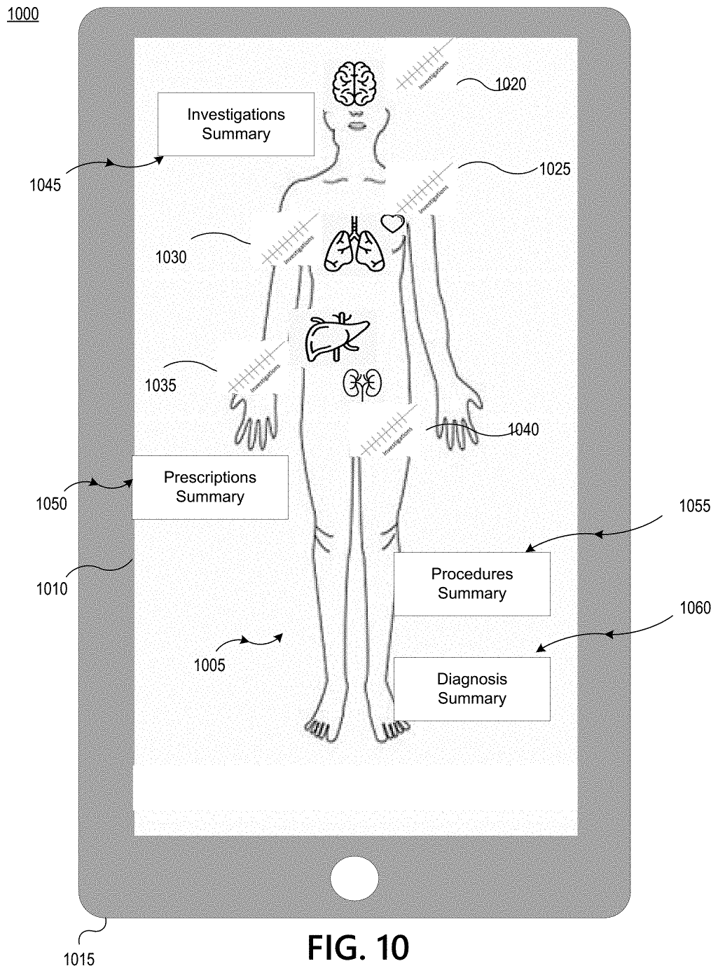

FIG. 10 depicts an illustrative frontal view 1000 for a real-time interactive digital embodiment of a patient in accordance with one or more example embodiments. Generally, the digital embodiment may be configured to display information from four Health Information Categories that are medically relevant, i.e., Investigations [Ix], Prescriptions [Rx], Diagnosis [Dx] and Procedure [Px] may be represented by icons (e.g., handbag, bangles, items of clothing etc.) on different parts of the body. Such a linkage between Health Information Category & icons associated with parts of the body (e.g., arms, legs, chest, back etc.) may be of significant utility. In some embodiments, real-time digital embodiment computing platform 110 may provide digital embodiment 1005 via a graphical user interface 1010 of a mobile device 1015.

As illustrated, digital embodiment 1005 may be associated with Investigations Summary 1045, Prescriptions Summary 1050, Procedures Summary 1055, and Diagnosis Summary 1060. Also, for example, various organs may be associated with various reports. For example, brain report 1020 may provide information associated with the brain, heart report 1025 may provide information associated with the heart, lung report 1030 may provide information associated with the lung, liver report 1035 may provide information associated with the liver, kidney report 1040 may provide information associated with the kidney, and so forth.

Upon selection of an icon for each Health Information category (e.g., Investigations [Ix] represented, for example, by a handbag in a left hand of the digital embodiment), information related to Investigations related from that time period may be provided from a cloud server to the digital embodiment, and displayed using a hierarchical tree structure inherent in the central database management (CDM).

Similarly, upon selection of another icon for another Health Information category (e.g., Prescriptions [Rx] represented by a medicine box in a right hand with a sign Rx displayed on it, as illustrated in FIGS. 8 and 9), information about prescription medications may be provided from the cloud server to the digital embodiment on the edge device (e.g., mobile device of the patient, mobile device of the medical provider), and these medications may also be displayed using hierarchical structures present in CDM (e.g., Atenolol is a Cardiac Drug→Anti-hypertensive→Beta blockers).

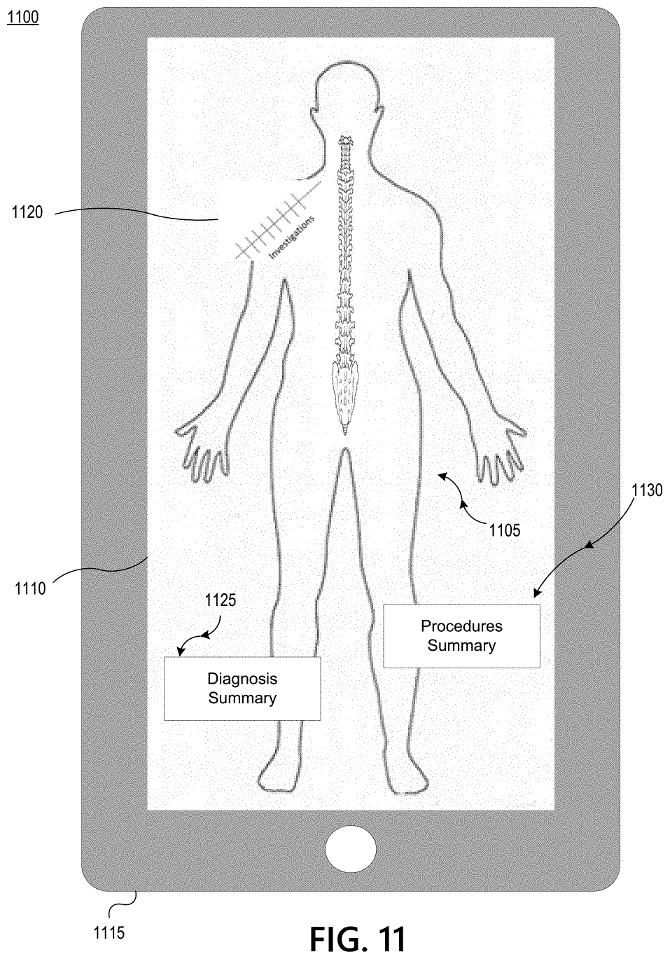

FIG. 11 depicts an illustrative dorsal view 1100 for a real-time interactive digital embodiment of a patient in accordance with one or more example embodiments. In some embodiments, real-time digital embodiment computing platform 110 may provide digital embodiment 1105 via a graphical user interface 1110 of a mobile device 1115. In some embodiments, real-time digital embodiment computing platform 110 may cause the display to move from a frontal view 1000 to a dorsal view 1100 based on receiving user indication to turn the digital embodiment.

As illustrated, digital embodiment 1105 may be associated with Procedures Summary 1130, and Diagnosis Summary 1125. Also, for example, spinal report 1120 may provide information associated with the spine. For example, radiological information associated with the spine may be provided. In some embodiments, digital embodiment 1105 may be configured to display spine deformities based on the radiological information. For example, if a surgical procedure was performed to fuse two vertebrae, real-time digital embodiment computing platform 110 may display the two vertebrae as fused together.

In some embodiments, real-time digital embodiment computing platform 110 may determine, based on health scores associated with organs of the patient, an aggregate health score for the patient. For example, the health scores associated with the patient may be added up to obtain the aggregate score. In some embodiments, the health scores may be weighted to obtain the aggregate scores. For example, certain health scores may be more significant for a certain age group, and such health scores may be assigned a greater weight. In some examples, the aggregate health score may be based on a mathematical relationship between the health scores.

Then, real-time digital embodiment computing platform 110 may determine, for the aggregate health score, an aggregate color for the digital embodiment, where the aggregate color is a combination of colors associated with the health scores. For example, the color scheme for the digital embodiment may range from a first color indicating that the patient is in good health, to a second color indicating that the patient is in poor health. Accordingly, a patient and a physician may be able to know the health of the patient from the color scheme. As may be noted, health scores may be updated in real-time or near real-time, and accordingly, the aggregate health score may be indicative of a current state of the patient's health.

FIG. 12 depicts an illustrative flow diagram for monitoring health attributes via a real-time interactive digital embodiment of a patient in accordance with one or more example embodiments. Referring to FIG. 12, at step 1205, a computing platform having at least one processor, a communication interface, and memory may identify medications being taken by a patient. At step 1210, the computing platform may determine a dosage for each medication. At step 1215, the computing platform may determine whether there is an interference between a first medication being taken by the patient, and a second medication being taken by the patient.

Upon a determination that there is no interference between a first medication being taken by the patient, and a second medication being taken by the patient, the process may proceed to step 1230. At step 1230, the computing platform may determine a time of dosage for each medication. The process may then proceed to step 1225.

Upon a determination that there is an interference between a first medication being taken by the patient, and a second medication being taken by the patient, the process may proceed to step 1220. At step 1220, the computing platform may determine a time of dosage for each medication to minimize or eliminate the interference between the first medication being taken by the patient and the second medication being taken by the patient. For example, based on information about a variety of prescribed medications, the computing platform may determine interferences between medicines and sets a lag time between different medicines to minimize interference. Then, the process may proceed to step 1225.

At step 1225, the computing platform may provide, to the patient and via the digital embodiment, a notification to take the medication at the determined time. At step 1235, the computing platform may determine whether the patient has indicated that the medication has been taken. For example, the patient may select an icon on a mobile application indicating that the medication has been taken.

At step 1235, upon a determination that the patient has not indicated that the medication has been taken, the process may proceed to step 1240. At step 1240, the computing platform may determine if a threshold has been exceeded. For example, the threshold may be a time threshold within which the dose of the medication needs to be taken. For example, if the time threshold is exceeded, the computing platform may infer that the patient may have missed the dose of the medication. Upon a determination that the time threshold has not been exceeded, the computing platform may send, to the patient and via the digital embodiment, a reminder to take the medication. Upon a determination that the time threshold has been exceeded, the process may proceed to step 1250.

As another example, the threshold may be a number of times a reminder is sent. For example, a limit of 3 reminders may be set, and at step 1240, the computing platform may determine if three reminders have been sent. Upon a determination that 1 or 2 reminders have been sent, the computing platform may send the next reminder. Upon a determination that 3 reminders have been sent, the computing platform may not send another reminder.

Generally, the loopback at steps 1235, 1240, 1245, and back to 1235, may be performed a predetermined number of times during a time threshold. For example, 3 reminders may be sent at 5, or 10, minute intervals.

At step 1235, upon a determination that the patient has indicated that the medication has been taken, the process may proceed to step 1250.

At step 1250, the computing platform may update the health attributes to indicate that the medication has been taken or has been missed. For example, if at step 1235, the computing platform determines that the patient has indicated that the medication has been taken, the computing platform may update the health attributes to indicate that the medication has been taken. Also, for example, if the computing platform determines, after a time threshold is exceeded, that the patient has not indicated that the medication has been taken, the computing platform may update the health attributes to indicate that the medication has been missed.

At step 1255, the computing platform may update the digital embodiment. For example, information associated with different Health Information Categories (e.g., Ix, Rx, Dx & Px) may be received from the patient. For example, information about missed medications may be provided via the digital embodiment. Such information may be displayed on time stamped digital embodiments, and a patient may be able to select an icon on the relevant digital embodiment, and such selection may trigger a scanning application to be initiated. The scanning application may enable the patient to capture a photograph of a report and/or prescription, and upload it to the cloud server. Based on such data, computing platform may apply one or more structuring algorithms to enter the information into the patients record at a CDM server.

A feedback feature may include, for example, providing a reminder, completion of a task to take the medication, acknowledgement/confirmation that task has been completed, and updating the digital embodiment. Such a feedback feature may alleviate issues related to a lack of compliance by a patient, which may be a significant reason as to why medications may not have their intended effect. As another example, a physician may now remotely know whether the patient is complying with the prescribed dosage.

FIG. 13 depicts another illustrative flow diagram for monitoring health attributes via a real-time interactive digital embodiment of a patient in accordance with one or more example embodiments. Referring to FIG. 13, at step 1305, a computing platform having at least one processor, a communication interface, and memory may identify a medication being taken by a patient. In some embodiments, the process may move to step 1310. At step 1310, the computing platform may determine whether the medication is associated with a required test.

Generally, there may be standard operating procedures for medications to have certain tests done. For example, if a patient is taking warfarin, which is a blood thinning medication, the patient may need to get a Prothrombin Time and International Normalized Ratio (PT/INR) test every month. Many physicians and/or patients may forget this test. There may be as many as 16000 medications in the database, and the computing platform may codify tests that may be mandatory for these medications, and such information may be utilized to configure the digital embodiment to provide appropriate reminders and/or notifications to patients and physicians for specific medicines prescribed to patients.

Upon a determination that the medication is associated with a required test, the process may proceed to step 1315. At step 1315, the computing platform may determine whether the rewuired test has been administered. Upon a determination that the test has been administered, the process may proceed to step 1320. At step 1320, the computing platform may display, via the digital embodiment, an indication for the physician. For example, the computing platform may display an indication that the test has been administered.

Upon a determination that the test has not been administered, the process may proceed to step 1330. At step 1330, the computing platform may generate, via the digital embodiment, an alert notification for the medical professional. At step 1325, the computing platform may determine whether a time threshold has been exceeded. Upon a determination that the time threshold has not been exceeded, the computing platform may return to step 1315. Upon a determination that the time threshold has been exceeded, the computing platform may proceed to step 1320. At step 1320, the computing platform may display, via the digital embodiment, an indication for the physician (e.g., via the physician's digital embodiment). For example, the computing platform may display an indication that the test has not been administered.

Upon a determination that the medication is not associated with a required test, the process may proceed to step 1335. In some embodiments, the process may proceed to step 1335 from step 1305. At step 1335, the computing platform may determine whether a quantity of medication consumed exceeds a dosage threshold. For example, medicines such as paracetamol, when taken in large quantities, may cause liver and/or kidney failure.

Upon a determination that the quantity of medication consumed exceeds the dosage threshold, the process may proceed to step 1340. At step 1340, the computing platform may display, via the digital embodiment, an indication for the patient (e.g., via the patient's digital embodiment). For example, the computing platform may display an indication to the patient that the dosage exceeds the dosage threshold, and further doses must be stopped, and/or recommend that the patient consult with their medical provider. In some embodiments, the process may proceed to step 1320. At step 1320, the computing platform may display an indication for the physician that the patient has exceeded their dosage threshold for the medication. For example, the indication may be a message, “5 gms. of paracetamol is the annual limit and the patient has already taken 5 gms.”

The digital embodiment comprising information for Ix, Rx, Dx & Px may be utilized effectively to highlight interactions at intra- and inter-level categories, especially interactions that may require medical attention. For instance, an adverse drug-drug interaction may be identified, and displayed to the doctor via the digital embodiment. Also, for example, based on summarized information from Ix and/or Dx, the computing platform may generate a recommendation for Rx and display such recommendation to the doctor. In general, information related to Ix, Rx, Dx, and Px may be synchronized (e.g., updated in real-time). In some embodiments, a holistic health view of the patient may be displayed via the digital embodiment. Accordingly, outliers may be identified and displayed to the doctor. Such timely recommendations may enable the doctor to take preventive, and/or remedial actions. Accordingly, the computing platform may monitor medication levels by tracking how much medicine has been consumed, what the safety level is, if the threshold has been reached or exceeded, and inform both patient and physician.

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored on a computer-readable medium such as a hard disk, optical disk, removable storage media, solid-state memory, RAM, and the like. The functionality of the program modules may be combined or distributed as desired in various embodiments.

Various aspects described herein may be embodied as a method, an apparatus, or as one or more computer-readable media storing computer-executable instructions. Accordingly, those aspects may take the form of an entirely hardware embodiment, an entirely software embodiment, an entirely firmware embodiment, or an embodiment combining software, hardware, and firmware aspects in any combination. In general, the one or more computer-readable media may comprise one or more non-transitory computer-readable media.

Numerous other embodiments, modifications, and variations within the scope and spirit of this disclosure will occur to persons of ordinary skill in the art from a review of this disclosure. For example, one or more of the steps may be performed in other than the recited order, and one or more steps may be optional in accordance with aspects of the disclosure.

What is claimed is:

1. A computing platform, comprising:
at least one processor;
a communication interface communicatively coupled to the at least one processor; and
memory storing computer-readable instructions that, when executed by the at least one processor, cause the computing platform to:
retrieve, via the communication interface and from a medical repository, an electronic health record associated with a patient;
extract, from the electronic health record, data indicative of:
a plurality of patient features indicative of one or more of a physical feature or a mental state associated with the patient; and
a plurality of health attributes of the patient;
configure a digital embodiment of the patient to:
display the plurality of patient features, and
display information associated with the plurality of health attributes;
render, via a graphical user interface of a computing device, the digital embodiment of the patient.

2. The computing platform of claim 1, wherein the instructions to configure the digital embodiment comprise further instructions that, when executed by the at least one processor, cause the computing platform to:
detect an interaction of the patient with a medical provider; and
apply a timestamp to the digital embodiment of the patient, wherein the timestamp is indicative of a time of the interaction.

3. The computing platform of claim 2, wherein the instructions to configure the digital embodiment comprise further instructions that, when executed by the at least one processor, cause the computing platform to:
configure, for each interaction of the patient with the medical provider, a temporal version of the digital embodiment, wherein the temporal version is indicative of the electronic health record at the time of the interaction.

4. The computing platform of claim 3, wherein the instructions to render the digital embodiment comprise further instructions that, when executed by the at least one processor, cause the computing platform to:
render, via the graphical user interface of the computing device, a plurality of temporal versions of the digital embodiment arranged in chronological order, wherein each temporal version of the plurality of temporal versions is associated with the time of the interaction.

5. The computing platform of claim 1, wherein the digital embodiment is a three-dimensional rendering of the patient.

6. The computing platform of claim 1, wherein the instructions comprise further instructions that, when executed by the at least one processor, cause the computing platform to:
detect, via the graphical user interface, a user interaction indicative of a movement associated with the digital embodiment; and
cause the digital embodiment to perform the indicated movement.

7. The computing platform of claim 1, wherein the computing device is associated with the patient, and wherein the instructions, when executed by the at least one processor, cause the computing platform to:
perform the generating based on one or more of a sub-plurality of the plurality of patient features, and a sub-plurality of the plurality of health attributes; and
provide the generated digital embodiment to the computing device associated with the patient.

8. The computing platform of claim 1, wherein the computing device is associated with a medical professional with an access to the electronic health record of the patient, and wherein the instructions, when executed by the at least one processor, cause the computing platform to:
perform the generating based on a sub-plurality of the plurality of patient features; and
provide the generated digital embodiment to the computing device associated with the medical professional.

9. The computing platform of claim 1, wherein the instructions to configure the digital embodiment comprise further instructions that, when executed by the at least one processor, cause the computing platform to:
identify, for a particular health attribute of the plurality of health attributes, a particular location on or around the digital embodiment corresponding to the particular health attribute; and
display the information associated with the particular health attribute at the particular location on the digital embodiment.

10. The computing platform of claim 1, wherein the information associated with the particular health attribute is located in a hierarchical level of a hierarchical structure of medical information.

11. The computing platform of claim 1, wherein the instructions to configure the digital embodiment comprise further instructions that, when executed by the at least one processor, cause the computing platform to:
detect a change in the electronic health record; and
update, based on the detected change, the rendering of the digital embodiment.

12. The computing platform of claim 1, wherein the instructions to configure the digital embodiment comprise further instructions that, when executed by the at least one processor, cause the computing platform to:
extract the plurality of patient features from a visual image or a video of the patient; and
configure the digital embodiment based on the extracted features.

13. The computing platform of claim 1, wherein the instructions to configure the digital embodiment comprise further instructions that, when executed by the at least one processor, cause the computing platform to:
animate a face of the digital embodiment to display one or more facial expressions.

14. The computing platform of claim 13, wherein the instructions to animate the face further comprise instructions that, when executed by the at least one processor, cause the computing platform to:
identify, for each facial expression, a collection of facial muscles associated with the facial expression;
associate, for the collection of facial muscles, a set of rules that mimic the facial expression on the face of the digital embodiment.

15. The computing platform of claim 1, wherein the instructions, when executed by the at least one processor, cause the computing platform to:
receive information related to a state of mind for the patient;
associate a facial expression with the state of mind; and
configure a face of the digital embodiment for the patient to display the associated facial expression.

16. The computing platform of claim 1, wherein the instructions, when executed by the at least one processor, cause the computing platform to:
associate, with the patient, a wellness score indicative of the patient's well-being;
associate, for the digital embodiment, a body posture with the wellness score; and
configure the body posture of the digital embodiment for the patient to display the wellness score.

17. The computing platform of claim 16, wherein the wellness score is received as an input from the patient.

18. The computing platform of claim 16, wherein the instructions, when executed by the at least one processor, cause the computing platform to:
associate, with each health attribute of the plurality of health attributes, an attribute score, and
wherein the wellness score is an aggregate of attribute scores.

19. The computing platform of claim 16, wherein the instructions, when executed by the at least one processor, cause the computing platform to:
determine, for each health attribute of the plurality of health attributes, a temporal trend;
associate, for the digital embodiment, a body posture with the temporal trend; and
configure the body posture of the digital embodiment for the patient to display the temporal trend.

20. The computing platform of claim 1, wherein the physical feature comprises one or more of hair color, eye color, eye movement, voice, gait, items of clothing, clothing accessories, and facial expression.

21. The computing platform of claim 1, wherein the instructions, when executed by the at least one processor, cause the computing platform to:
update, in real-time, the rendering of the digital embodiment.

22. The computing platform of claim 1, wherein the instructions, when executed by the at least one processor, cause the computing platform to:
associate, with each organ of the patient and based on the electronic health record, a health score indicative of a health of the organ;
associate, with each health score, a color scheme;
determine, for each organ of the patient, a region of the digital embodiment associated with the organ; and
display, for the region and based on the health score associated with the organ, a color from the color scheme.

23. The computing platform of claim 22, wherein the instructions, when executed by the at least one processor, cause the computing platform to:
determine, based on health scores associated with organs of the patient, an aggregate health score for the patient; and
determine, for the aggregate health score, an aggregate color for the digital embodiment, wherein the aggregate color is a combination of colors associated with the health scores.

24. The computing platform of claim 1, wherein the instructions, when executed by the at least one processor, cause the computing platform to:
detect, from the electronic health record, presence of a medical implant in the patient;
determine, from the electronic health record, a physical location of the medical implant; and
configure the interactive digital embodiment of the patient to display an indication of the medical implant at a location, on the digital embodiment, that corresponds to the physical location.

25. A method, comprising:
at a computing platform comprising at least one processor, a communication interface, and memory:
retrieving, via the communication interface and from a medical repository, an electronic health record associated with a patient;
extracting, from the electronic health record:
a plurality of patient features indicative of one or more of a physical feature or a mental state associated with the patient, and
a plurality of health attributes of the patient;
configuring an interactive digital embodiment of the patient to:
display the plurality of patient features, and
display information associated with the plurality of health attributes;
rendering, via a graphical user interface of a computing device, the interactive digital embodiment of the patient.

26. One or more non-transitory computer-readable media storing instructions that, when executed by a computing platform comprising at least one processor, a communication interface, and memory, cause the computing platform to:
retrieve, via the communication interface and from a medical repository, electronic health record associated with a patient;
extract, from the electronic health record:
a plurality of patient features indicative of one or more of a physical feature or a mental state associated with the patient, and
a plurality of health attributes of the patient;
configure a digital embodiment of the patient to:
display the plurality of patient features, and
display information associated with the plurality of health attributes;
render, via a graphical user interface of a computing device, the digital embodiment of the patient.
